(19) United States
(12) Patent Application Publication
(10) Pub. No.: US 2014/0249466 A1
(43) Pub. Date: Sep. 4, 2014

(54) SYSTEM AND METHOD FOR NEUROMODULATION OF BODY TEMPERATURE REGULATION SYSTEM

(71) Applicant: Gil Hakim, Ra'anana (IL)

(72) Inventor: Gil Hakim, Ra'anana (IL)

(73) Assignee: THERMACON LTD., Yokneam Ilit (IL)

(21) Appl. No.: 14/126,024

(22) PCT Filed: Jun. 14, 2012
(86) PCT No.: PCT/IL2012/000235

§ 371 Date: May 8, 2014

Related U.S. Application Data
(60) Provisional application No. 61/496,588, filed on Jun. 14, 2011.

(51) Int. Cl. A61N 1/36; A61F 7/02; A61M 35/00

(52) U.S. Cl. CPC A61N 1/36017; A61M 35/00; A61F 7/02
USPC 604/20; 607/62; 607/59; 607/3

(57) ABSTRACT

A system for controlling and modulating a mammalian's body temperature and thermoregulation system includes: a. a display monitor; b. a pulse generator; c. at least one channel of electrical or electromagnetic stimuli connected to the pulse generator, and adapted for cutaneous application on the body's spinal cord; d. a control panel, and e. at least one Measurement Analysis and Command (MAC) unit. The control panel and the MAC units are adapted to implement the optimal stimulating procedure for blocking the afferent neural pathways, evoking the afferent neural pathways, attenuating the afferent neural pathways, blocking and evoking the efferent neural pathways, blocking and attenuating the efferent neural pathways, attenuating and evoking, or blocking, attenuating, and evoking the efferent neural pathways, involved in the thermoregulation system, thereby controlling and modulating the body's temperature and thermoregulation system.

### BACKGROUND OF THE INVENTION

The present invention relates generally to a system and method for a manipulation of the thermoregulation of a mammalian body's temperature.

Temperature management of the body is crucial to patient lives. Optimizing temperature management improves patient outcome and reduces health care cost.

The natural body thermoregulation system is a complicated mechanism for maintaining stable core body temperature. The hypothalamus contains control mechanisms and key temperature sensors. Under control of these mechanisms, sweating begins almost precisely at a skin temperature of 37° C. and increases rapidly as the skin temperature rises above this value. The heat production of the body under these conditions remains almost constant as the skin temperature rises. If the skin temperature drops below 37° C. a variety of responses are initiated to conserve the heat in the body and to increase heat production. These include vasoconstriction to decrease the flow of heat to the skin, cessation of sweating, shivering to increase heat production in the muscles, and secretion of norepinephrine, epinephrine and thyroxine to increase heat production. Messages from the brain reach effectors. For example, the trigger to shivering is correlated to core temperature drop. Messages reach muscles and glands, via the motor neurons.

During surgical operations, the body's core temperature can drop due to the re-distribution of heat from the body's core to peripheral tissue, due to the vasodilating effect of anesthesia drugs and due the lack of protective vasoconstriction. Many patients, about 70%, become hypothermic, post surgery. Hypothermia becomes clinically significant in operations longer than two hours and leads to increased patient morbidity and healthcare costs.

In some clinical indications however, where there is ischemic stress or other stresses such as toxic metabolites in the brain, the heart, kidney or liver, such as a stroke, Cardiac Arrest (CA), Traumatic Brain Injury (TBI), Acute Myocardial Infarction (AMI), Contrast induced nephropathy, Acetaminophen-induced liver failure, the intentional induction of hypothermia was found to be a therapeutic option. The benefits of cooling were found to be greater than the risks, and it was shown that the chance for the patient's of recovery is increased.

For example, current devices use external heat exchange apparatus without coping with the body's thermoregulation physiological responses. Other devices and methods are invasive and complicated, and can be applied only at the hospital's emergency room, and critical time may be lost.

External heat exchange modules or energy affects the peripheral thermal sensors. Heat is transferred in or out via convection, through the blood. Drugs are used to modulate the thermoregulation system. Heat transfer is restricted due to limits in temperature level that may be safely applied and thermoregulation system resistance.

As disclosed in the following description, there are many known methods for controlling the body's thermoregulation system using: heat transfer or administration of medication. However, there is still a significant need to provide means for non-invasively controlling the main thermostat system of the body, without cumbersome external heat/cooling transfer or medication.

Dobak U.S. Pat. No. 6,364,899 offers a solution for pain control and discloses a heat pipe to a spinal cord of a patient, for producing reversible focal hypothermia of the nervous system to control chronic pain. Nerve conduction is blocked by mild cooling, or hypothermia.

Holsheimer U.S. Pat. No. 5,643,330 discloses an apparatus for providing a number of superimposed current generated electrical fields for epidural spinal cord stimulation. The pulses given, by the stimulator channels, are selectably simultaneous or alternate in time, are selectably equal or different in amplitude, or both, permitting the shifting the electrical field after implantation to optimize paresthesia effects (abnormal sensation, imaginary sensation) or to eliminate unwanted motor responses.

Dae U.S. Pat. No. 6,572,638 discloses a method and apparatus for controlling the body temperature of a patient, while reducing shivering by using a heat exchange device in combination with an anti-thermoregulatory response mechanism including various anti-thermoregulatory response agents that temporarily reduce shivering. The devices disclosed include a catheter having a heat exchange balloon thereon with heat exchange fluid circulating through the interior of the balloon, placed in the vasculature of a patient, in order to add or remove heat from the blood of the patient. The system includes a control for controlling the patient's temperature in conjunction with administering the anti-thermoregulatory response mechanism.

Diller US20110066217 discloses a method for increasing or maintaining temperature of glabrous tissue in a subject, by applying heat to peripheral thermoregulatory control tissue of the subject, where the applied heat increases or maintains perfusion of blood in the glabrous tissue; method further includes negative pressure to the glabrous tissue. Said peripheral thermoregulatory control tissue is located in the cervical spinal region or in the lumbar spinal region of the subject. In other aspects, Diller discloses cooling stimulus that can be applied to the glabrous tissue with the increased or maintained perfusion. When a cooling stimulus is used, the core temperature of the subject can be reduced. When a warming stimulus is used, the core temperate of the subject can be increased.

Carson U.S. Pat. No. 6,799,063 discloses a dual function medical pad is disclosed for both controlling patient temperature and providing a patient-to-electrode interface. The pad includes a fluid containing layer for containing a thermal exchange fluid circulated there through, operable for thermal exchange with a patient. Further electrodes interconnected to the fluid containing layer, such as electrosurgical return electrodes, EKG electrodes, pacing or defibrillation electrodes.

Grahn U.S. Pat. No. 7,947,068 discloses a method for controlling the temperature of all or a portion of a patient's body, while achieving thermoregulatory inhibition. The method includes sensing the temperature of all or a portion of the patient's body and controlling the temperature of all or a portion of the patient's body based upon the signal by placing a heat exchange device having a heat exchange region into heat exchange proximity with the patient's body and controlling the temperature of the heat exchange region for a sufficient time to affect the temperature of all or a portion of the patient's body; and further by administering a therapeutically effective amount of an anti-thermoregulatory response agent to the patient for the inactivation of the shivering response and for the inhibition of vasoconstriction.

Flint US20100087900 discloses a method for quantifying shivering in a subject, by obtaining and analyzing signals from a muscle mass that is susceptible to shivering; the signals include an ECG component and an EMG component. Flint further discloses a method for treating shivering in a subject in the course of therapeutic temperature regulation for inhibition of shivering, to areas such as: hands, feet, ears, upper back or posterior neck. Flint further discloses a method for applying active counterwarming to a subject during therapeutic temperature regulation for a patient; cooling apparatus may include a cooling blanket, cooling pads, and an endovascular cooling catheter.

Pan-Li CN101920066 discloses a wearable electrical stimulation system for wearing on a shivering limb, for pathological tremor suppression. The system includes: a wearable binding structure, an electromyographic sensing device, an information processor, a controller and an actuator.

Louise US20110125205 discloses a thermo-stimulation system for providing electrical currents for thermal and electrical stimulation including a plurality of thermo-stimulation pads regulated in response to a temperature feedback by an inline control system. The system is designed for a variety of therapeutic applications, such as heat therapy and thermo-stimulation using electrical stimulation application a single or a group of muscles. The resulting contraction can produces a variety of effects from strengthening injured muscles and reducing oedema to relieving pain and promoting healing.

It is clear from the above that there remains a long felt and unmet need to provide improved and more comprehensive means and methods of artificially controlling the body temperature of a patient in need. The present invention as will be further detailed in the description, discloses a novel system for manipulating the body thermoregulation system, without the use of a thermal stimuli for body temperature control, as seen in the prior art.

### SUMMARY OF THE INVENTION

It is one object of the present invention to provide a system [1] for controlling and modulating a mammalian's body temperature and thermoregulation system, comprising:
a. a display monitor [30];
b. a pulse generator [10];
c. at least one channel of electrical or electromagnetic stimuli configured by at least one pair of stimulating electrodes [5], connected to said pulse generator [10], and adapted for cutaneous application on said body's spinal cord, transcutaneously or by minimally invasive means;
d. a control panel [20], for controlling said pulse generator [10] and said at least one pair of stimulating electrodes [5], and
e. at least one Measurement Analysis and Command (MAC) unit for measuring physiological parameters, analyzing their signals and commanding an optimal stimulating procedure to said control panel [20];
   wherein said control panel [20] and said MAC units are adapted to implement said optimal stimulating procedure for blocking the afferent neural pathways, evoking said afferent neural pathways, attenuating said afferent neural pathways, blocking and evoking said efferent neural pathways, blocking and attenuating said efferent neural pathways, attenuating and evoking, or blocking, attenuating, and evoking said efferent neural pathways, involved in said thermoregulation system, thereby controlling and modulating said body's temperature and thermoregulation system.

It is another object of the present invention to provide the system [1] as defined above, wherein said a pulse generator [10] is selected from the group consisting of:
a. Trans Electrical Neuro Stimulation (TENS)
b. Interferential Current (IFC)
c. Electro Magnetic stimulation (EMS)
or any combination thereof.

It is another object of the present invention to provide the system [1] as defined above, wherein said MAC unit is at least one unit selected from the group consisting of:
a. a temperature (T) unit [70], used for measuring skin temperature, core temperature, or skin and core temperature;
b. a Heart Rate Variability (HRV) unit [50], used for measuring heart beat;
c. an Electromyography (EMG) unit [60], used for measuring muscles electrical activity;
d. an oxygen utilization (Vo2) measuring unit [80], for measuring oxygen utilization, and
e. a neuro-signal unit [90], used for measuring neuro-electric activity;
or any combination thereof;
   said MAC units adapted to receive and analyze said measured signals and to command the administration of said optimal pulse stimulating procedure to said control panel [20].

It is another object of the present invention to provide the system [1] as defined above, wherein said MAC units are further adapted to analyze said measured physiological signals as feedback signals for monitoring said applied pulse stimulating procedure, and command said control panel [20] accordingly.

It is another object of the present invention to provide the system [1] as defined above, wherein said control panel [20] and said MAC units are adapted to command at least one predetermined pulse stimulating procedure selected from the group consisting of:
stimulating the fast conduction Aβ fibers, thereby modulating the “thermal input” transferred to the hypothalamus;
stimulating said Aβ fibers for controlling the passage of sensory information traveling to said hypothalamus;
stimulating the slow conduction C fibers for a “heat” input, thereby modulating said thermal input received by said hypothalamus;
stimulating said C fibers for blocking said “heat” input, thereby modulating said thermal input received by said hypothalamus and avoiding new action potential to be generated;
stimulating the medium conduction Aδ fibers for a “cold” input message, thereby modulating said thermal input received by said hypothalamus;
stimulating said Aδ fibers for blocking the “cold” input message, thereby modulating said thermal input received by said hypothalamus and avoiding new action potential to be generated;
stimulation or blockage of efferent motor neurons receiving input from the thermoregulation system, related to changes in body temperature, thereby modulating said motor neurons activity;
stimulating the nerve fibers to simulate “cold” or “heat” input, from different parts of said body;
or any combination thereof.

It is another object of the present invention to provide the system [1] as defined above, wherein said control panel [20] and said MAC units regulate said stimulating procedures of electric pulses by means of current's amplitude [A], frequency [F] and phase [φ], applied to said selected channels of electrical or electromagnetic stimuli.

It is another object of the present invention to provide the system [1] as defined above, wherein said control panel [20] and said MAC units are configured to regulate said frequency [F] in the range of about 0 to about 30,000 [Hz].

It is another object of the present invention to provide the system [1] as defined above, wherein said control panel [20] and said MAC units are configured to regulate said frequencies' [F] beat in the range of about 0 to about 1000 [Hz].

It is another object of the present invention to provide the system [1] as defined above, wherein said control panel [20] and said MAC units are configured to implement said stimulating procedures in cycles which said frequencies' [F] beat are not constant between cycle to cycle.

It is another object of the present invention to provide the system [1] as defined above, wherein said control panel [20] and said MAC units are configured to regulate said frequency [F] in a manner selected from the group consisting of: linear, nonlinear, sinusoidal, or any combination thereof.

It is another object of the present invention to provide the system [1] as defined above, wherein said control panel [20] and said MAC units are configured to implement said stimulating procedures in intervals which range from about 0 to about 300 [sec].

It is another object of the present invention to provide the system [1] as defined above, wherein said control panel [20] and said MAC units are adapted to implement said pulse stimulating procedure for blocking the temperature signals traveling from the entire body or selected body parts from reaching the brain, thereby isolating the thermoregulation system from reacting to changes in skin or core temperature

It is another object of the present invention to provide the system [1] as defined above, wherein said control panel [20] and said MAC units are adapted to implement said stimulating procedure adapted to control and modulate said body's thermoregulation system to at least one of the following:
a. maintain normo-thermia or sensation of normo-thermia;
b. either induce or maintain hypo-thermia or sensation of hypo-thermia;
c. either induce or maintain hyper-thermia or sensation of hyper-thermia.

It is another object of the present invention to provide the system [1] as defined above, wherein said control panel [20] and said MAC units are adapted to implement said stimulating procedure adapted to control and modulate said thermoregulation system physiological responses such as: vasodilatation, vasoconstriction of blood vessels, shivering or heart rate, by affecting the efferent neurological pathways.

It is another object of the present invention to provide the system [1] as defined above, wherein said at least one pair of stimulating electrodes [5] are configured for predetermined location selected from the group consisting of: topically close to the spinal cord, remote from said spinal cord, transcutaneously, or by means of minimally invasive means to said spinal cord, or any combination thereof.

It is another object of the present invention to provide the system [1] as defined above, wherein said at least one pair of stimulating electrodes [5] are configured to be attached to said body using at least one patch [6].

It is another object of the present invention to provide the system [1] as defined above, wherein said at least one pair of stimulating electrodes [5] are further configured to operate as sensors for measuring Electro-Cardio-Graph (ECG) signals.

It is another object of the present invention to provide the system [1] as defined above, wherein said least one pair of stimulating electrodes [5] are further configured to operate as sensors for measuring Neuro-Electric signals.

It is another object of the present invention to provide the system [1] as defined above, wherein said control panel [20] and said MAC units are adapted to command said stimulation procedure along the whole length of the spinal cord or selected segments thereof, according to required treatment and clinical need.

It is another object of the present invention to provide the system [1] as defined above, wherein said control panel [20] and said MAC units are adapted to command and implement said stimulation procedure for controlling said body's temperature and thermoregulation system for the entire body or selected body parts.

It is another object of the present invention to provide the system [1] as defined above, wherein said control panel [20] and said MAC units are adapted to command said stimulation procedure for simultaneously controlling said selected body parts, each for normo-thermia, hyper-thermia or hypo-thermia.

It is another object of the present invention to provide the system [1] as defined above, wherein said system [1] further comprises means for the application of medication such as: local or general anesthetics, analgesics and or sedation agents, muscle blocking or relaxants agents, pain blocking agents, consciousness impairing agents.

It is another object of the present invention to provide the system [1] as defined above, wherein said control panel [20] controls said application of stimulation procedure automatically.

It is another object of the present invention to provide the system [1] as defined above, wherein said control panel [20] controls said application of stimulation procedures at least partially manually.

It is another object of the present invention to provide the system [1] as defined above, wherein said display monitor [30] is configured to display parameters selected from the group consisting of: said MAC units measurements, said MAC units analysis results, said MAC units command for said stimulating procedures, said control panel [20] optional said stimulating procedures, said control panel [20] said administrated stimulating procedure, or any combination thereof.

It is another object of the present invention to provide the system [1] as defined above, wherein said control panel [20] and said display monitor [30] are combined into a single device, such as a touch screen.

It is another object of the present invention to provide the system [1] as defined above, wherein said control panel [20], said display monitor [30] and said pulse generator [10] are combined into a single device.

It is another object of the present invention to provide the system [1] as defined above, wherein said at least two MAC units are activated simultaneously, said control panel [20] consequent upon the first MAC unit that requires a change in generation of said pulse stimulating procedure.

It is another object of the present invention to provide the system [1] as defined above, wherein said HRV unit [50] further comprises: an HRV receiving unit [51], an HRV analysis unit [52], an HRV activating unit [53] and an HRV output pulse generator [59].

It is another object of the present invention to provide the system [1] as defined above, wherein said EMG unit [60] further comprises: an EMG receiving unit [61], an EMG analysis unit [62], an EMG activating unit [63], an EMG identification unit [65] and an EMG output pulse generator [69], as shown in FIG. 5.

It is another object of the present invention to provide the system [1] as defined above, wherein said temperature measuring unit [70] further comprises: a skin receiving unit [71s], a core receiving unit [71c], a temperature measuring analysis unit [62], a temperature measuring activating unit [73] and a temperature measuring output pulse generator [79], as shown in FIG. 6.

It is another object of the present invention to provide the system [1] as defined above, wherein said oxygen utilization (Vo2) measuring unit [80] further comprises: a Vo2 receiving unit [81], a Vo2 analysis unit [82], a Vo2 activating unit [83] and a Vo2 output pulse generator [89], as shown in FIG. 7.

It is another object of the present invention to provide the system [1] as defined above, wherein said neuro-signal unit [90] further comprises: a neuro-signal receiving unit [91], a neuro-signal analysis unit [92], a neuro-signal activating unit [93], a neuro-signal identification unit [95], a neuro-signal summing unit [96] and a neuro-signal output pulse generator [99], as shown in FIG. 8.

It is another object of the present invention to provide the system [1] as defined above, wherein said system [1] further comprises at least one Heat Exchange Unit (HEU) [100] for heating or cooling said body.

It is another object of the present invention to provide the system [1] as defined above, wherein said HEU [100] further comprises: at least one thermal sensor [101], an HEU analysis unit [102], an HEU activating unit [103] and at least one of the following:
an HEU output to heaters unit [109] and at least one heater [105]
an HEU output to coolers unit and at least one cooler.

It is another object of the present invention to provide the system [1] as defined above, wherein said heaters [105] or said coolers are placed on a body part selected from the group consisting of hand palms, feet soles, wrapped around large body parts, or any combination thereof.

It is another object of the present invention to provide the system [1] as defined above, wherein said least one pair of stimulating electrodes [5] are configured to be placed parallel to the vertebral column and over the intervertebral foramen, thereby enabling stimulation of the appropriate roots of spinal nerves which supply the affected dermatome.

It is another object of the present invention to provide the system [1] as defined above, wherein said least one pair of stimulating electrodes [5] layout in relation to the spinal cord vertebrates in a configuration selected from the group consisting of:
a. in a perpendicular manner, between said vertebrates as shown in FIG. 10 A;
b. in a perpendicular manner, across said vertebrates as shown in FIG. 10 B;
c. in a cross diagonally manner, where one electrode is near one vertebrate and the other electrode is near the second following vertebrate, as shown in FIG. 10 C;
d. in a cross diagonally manner, between said vertebrates, where one is close to said spine and the other is far from said spine, as shown in FIG. 10 D;
e. in a cross diagonally manner, between the vertebrates, as shown in FIG. 10 E;
f. in a cross diagonally manner, where one electrode is near one vertebrate and the other electrode is near the following vertebrate, as in FIG. 10 F;
g. in a cross diagonally manner, between said vertebrates, as shown in FIG. 10 G;
h. in a cross diagonally manner, between said vertebrates, where both said electrodes are on the same side of said spine, as shown in FIG. 10 H;
i. in a cross diagonally manner, where one electrode is near one vertebrate and the other electrode is far from the second following vertebrate, as shown in FIG. 10 I;
or any combination thereof.

It is another object of the present invention to provide a method for controlling and modulating a mammalian's body temperature and thermoregulation system, said method comprising steps of
a. obtaining a system [1] for controlling and modulating a mammalian's body temperature and thermoregulation system, comprising:
   i. a display monitor [30];
   ii. a pulse generator [10];
   iii. at least one channel or electrical or electromagnetic stimuli configured by at least one pair of stimulating electrodes [5], connected to said pulse generator [10], and adapted for cutaneous application on said body's spinal cord, transcutaneously or by minimally invasive means;
   iv. a control panel [20], for controlling said pulse generator [10] and said at least one pair of stimulating electrodes [5], and
   v. at least one Measurement Analysis and Command (MAC) unit for measuring physiological parameters, analyzing their signals and commanding an optimal pulse stimulating procedure to said control panel [20];
b. placing said electrodes over the skin bilaterally along said body's back;
c. measuring said physiological parameters;
d. receiving said measured physiological parameters;
e. analyzing said measured physiological parameters;
f. determining whether a change in the parasympathetic activity in the body requires activating the pulse generator [10], due to a change in the patient's body temperature;
g. determining which of said electrodes [5], placed on which dermatome, should be stimulated;
h. determining an optimal stimulation procedure;
i. stimulating said optimal stimulation procedure, at below pain threshold level, thereby inducing sense of heat or coldness;
j. stimulating said optimal stimulation procedure, for covering all somatic dermatomes, such that all incoming thermal data is subject to filtering or modulation, and
k. monitoring said measured physiological parameters as feedback signals for monitoring said applied stimulating procedure, and commanding said control panel [20] accordingly;
wherein said optimal pulse stimulating procedure is adapted to block the afferent neural pathways, evoke said afferent neural pathways, attenuate said afferent neural pathways, block and evoke said efferent neural pathways, block and attenuate said efferent neural pathways, attenuate and evoke, or block, attenuate, and evoke said efferent neural pathways, involved in said thermoregulation system, thereby controlling and modulating said body's temperature and thermoregulation system.

It is another object of the present invention to provide the method as defined above, wherein said method further comprises the step of analyzing the input signals from said HRV unit in time and in frequency domains.

It is another object of the present invention to provide the method as defined above, wherein said a pulse generator [10] is selected from the group consisting of:
a. Trans Electrical Neuro Stimulation (TENS)
b. Interferential Current (IFC)
c. Electro Magnetic stimulation (EMS)
or any combination thereof.

It is another object of the present invention to provide the method as defined above, wherein said MAC unit is at least one unit selected from the group consisting of:
a. a temperature (T) unit [70], used for measuring skin temperature, core temperature, or skin and core temperature;
b. a Heart Rate Variability (HRV) unit [50], used for measuring heart beat;
c. an Electromyography (EMG) unit [60], used for measuring muscles electrical activity;
d. an oxygen utilization (Vo2) measuring unit [80], for measuring oxygen utilization and
e. a neuro-signal unit [90], used for measuring neuro-electric activity;
or any combination thereof;
   said MAC units adapted to receive and analyze said measured signals and to command the administration of said optimal pulse stimulating procedure to said control panel [20].

It is another object of the present invention to provide the method as defined above, wherein said MAC units are further adapted to analyze said measured physiological signals as feedback signals for monitoring said applied pulse stimulating procedure, and command said control panel [20] accordingly.

It is another object of the present invention to provide the method as defined above, wherein said control panel [20] and said MAC units are adapted to command at least one predetermined pulse stimulating procedure selected from the group consisting of:
stimulating the fast conduction Aβ fibers, thereby modulating the “thermal input” transferred to the hypothalamus;
stimulating said Aβ fibers for controlling the passage of sensory information traveling to said hypothalamus;
stimulating the slow conduction C fibers for a “heat” input, thereby modulating said thermal input received by said hypothalamus;
stimulating said C fibers for blocking said “heat” input, thereby modulating said thermal input received by said hypothalamus and avoiding new action potential to be generated;
stimulating the medium conduction Aδ fibers for a “cold” input message, thereby modulating said thermal input received by said hypothalamus;
stimulating said Aδ fibers for blocking the “cold” input message, thereby modulating said thermal input received by said hypothalamus and avoiding new action potential to be generated;
stimulation or blockage of efferent motor neurons receiving input from the thermoregulation system, related to changes in body temperature, thereby modulating said motor neurons activity;
stimulating the nerve fibers to simulate “cold” or “heat” input, from different parts of said body;
or any combination thereof.

It is another object of the present invention to provide the method as defined above, wherein said control panel [20] and said MAC units regulate said stimulating procedures of electric pulses by means of current's amplitude [A], frequency [F] and phase [φ], applied to said selected channels of electrical or electromagnetic stimuli.

It is another object of the present invention to provide the method as defined above, wherein said control panel [20] and said MAC units are configured to regulate said frequency [F] in the range of about 0 to about 30,000 [Hz].

It is another object of the present invention to provide the method as defined above, wherein said control panel [20] and said MAC units are configured to regulate said frequencies' [F] beat in the range of about 0 to about 1000 [Hz].

It is another object of the present invention to provide the method as defined above, wherein said control panel [20] and said MAC units are configured to implement said stimulating procedures in cycles which said frequencies' [F] beat are not constant between cycle to cycle.

It is another object of the present invention to provide the method as defined above, wherein said control panel [20] and said MAC units are configured to regulate said frequency [F] in a manner selected from the group consisting of: linear, nonlinear, sinusoidal, or any combination thereof.

It is another object of the present invention to provide the method as defined above, wherein said control panel [20] and said MAC units are configured to implement said stimulating procedures in intervals which range from about 0 to about 300 [sec].

It is another object of the present invention to provide the method as defined above, wherein said control panel [20] and said MAC units are adapted to implement said pulse stimulating procedure for blocking the temperature signals traveling from the entire body or selected body parts from reaching the brain, thereby isolating the thermoregulation system from reacting to changes in skin or core temperature

It is another object of the present invention to provide the method as defined above, wherein said control panel [20] and said MAC units are adapted to implement said stimulating procedure adapted to control and modulate said body's thermoregulation system to at least one of the following:
a. maintain normo-thermia or sensation of normo-thermia;
b. either induce or maintain hypo-thermia or sensation of hypo-thermia;
c. either induce or maintain hyper-thermia or sensation of hyper-thermia.

It is another object of the present invention to provide the method as defined above, wherein said control panel [20] and said MAC units are adapted to implement said stimulating procedure adapted to control and modulate said thermoregulation system physiological responses such as: vasodilatation, vasoconstriction of blood vessels, shivering or heart rate, by affecting the efferent neurological pathways.

It is another object of the present invention to provide the method as defined above, wherein said at least one pair of stimulating electrodes [5] are configured for predetermined location selected from the group consisting of: topically close to the spinal cord, remote from said spinal cord, transcutaneously, or by means of minimally invasive means to said spinal cord, or any combination thereof.

It is another object of the present invention to provide the method as defined above, wherein said at least one pair of stimulating electrodes [5] are configured to be attached to said body using at least one patch [6].

It is another object of the present invention to provide the method as defined above, wherein said at least one pair of stimulating electrodes [5] are further configured to operate as sensors for measuring Electro-Cardio-Graph (ECG) signals.

It is another object of the present invention to provide the method as defined above, wherein said least one pair of stimulating electrodes [5] are further configured to operate as sensors for measuring Neuro-Electric signals.

It is another object of the present invention to provide the method as defined above, wherein said control panel [20] and said MAC units are adapted to command said stimulation procedure along the whole length of the spinal cord or selected segments thereof, according to required treatment and clinical need.

It is another object of the present invention to provide the method as defined above, wherein said control panel [20] and said MAC units are adapted to command and implement said stimulation procedure for controlling said body's temperature and thermoregulation system for the entire body or selected body parts.

It is another object of the present invention to provide the method as defined above, wherein said control panel [20] and said MAC units are adapted to command said stimulation procedure for simultaneously controlling said selected body parts, each for normo-thermia, hyper-thermia or hypo-thermia.

It is another object of the present invention to provide the method as defined above, wherein said system [1] further comprises means for the application of medication such as: local or general anesthetics, analgesics and or sedation agents, muscle blocking or relaxants agents, pain blocking agents, consciousness impairing agents.

It is another object of the present invention to provide the method as defined above, wherein said control panel [20] controls said application of stimulation procedure automatically.

It is another object of the present invention to provide the method as defined above, wherein said control panel [20] controls said application of stimulation procedures at least partially manually.

It is another object of the present invention to provide the method as defined above, wherein said display monitor [30] is configured to display parameters selected from the group consisting of: said MAC units measurements, said MAC units analysis results, said MAC units command for said stimulating procedures, said control panel [20] optional said stimulating procedures, said control panel [20] said administrated stimulating procedure, or any combination thereof.

It is another object of the present invention to provide the method as defined above, wherein said control panel [20] and said display monitor [30] are combined into a single device, such as a touch screen.

It is another object of the present invention to provide the method as defined above, wherein said control panel [20], said display monitor [30] and said pulse generator [10] are combined into a single device.

It is another object of the present invention to provide the method as defined above, wherein said at least two MAC units are activated simultaneously, said control panel [20] consequent upon the first MAC unit that requires a change in generation of said pulse stimulating procedure.

It is another object of the present invention to provide the method as defined above, wherein said HRV unit [50] further comprises: an HRV receiving unit [51], an HRV analysis unit [52], an HRV activating unit [53] and an HRV output pulse generator [59].

It is another object of the present invention to provide the method as defined above, wherein said EMG unit [60] further comprises: an EMG receiving unit [61], an EMG analysis unit [62], an EMG activating unit [63], an EMG identification unit [65] and an EMG output pulse generator [69], as shown in FIG. 5.

It is another object of the present invention to provide the method as defined above, wherein said temperature measuring unit [70] further comprises: a skin receiving unit [71s], a core receiving unit [71c], a temperature measuring analysis unit [62], a temperature measuring activating unit [73] and a temperature measuring output pulse generator [79], as shown in FIG. 6.

It is another object of the present invention to provide the method as defined above, wherein said oxygen utilization (Vo2) measuring unit [80] further comprises: a Vo2 receiving unit [81], a Vo2 analysis unit [82], a Vo2 activating unit [83] and a Vo2 output pulse generator [89], as shown in FIG. 7.

It is another object of the present invention to provide the method as defined above, wherein said neuro-signal unit [90] further comprises: a neuro-signal receiving unit [91], a neuro-signal analysis unit [92], a neuro-signal activating unit [93], a neuro-signal identification unit [95], a neuro-signal summing unit [96] and a neuro-signal output pulse generator [99], as shown in FIG. 8.

It is another object of the present invention to provide the method as defined above, wherein said system [1] further comprises at least one Heat Exchange Unit (HEU) [100] for heating or cooling said body.

It is another object of the present invention to provide the method as defined above, wherein said HEU [100] further comprises: at least one thermal sensor [101], an HEU analysis unit [102], an HEU activating unit [103] and at least one of the following:
an HEU output to heaters unit [109] and at least one heater [105]
an HEU output to coolers unit and at least one cooler.

It is another object of the present invention to provide the method as defined above, wherein said heaters [105] or said coolers are placed on a body part selected from the group consisting of: hand palms, feet soles, wrapped around large body parts, or any combination thereof.

It is another object of the present invention to provide the method as defined above, wherein said least one pair of stimulating electrodes [5] are configured to be placed parallel to the vertebral column and over the intervertebral foramen, thereby enabling stimulation of the appropriate roots of spinal nerves which supply the affected dermatome.

It is another object of the present invention to provide the method as defined above, wherein said least one pair of stimulating electrodes [5] layout in relation to the spinal cord vertebrates in a configuration selected from the group consisting of:
a. in a perpendicular manner, between said vertebrates as shown in FIG. 10 A;
b. in a perpendicular manner, across said vertebrates as shown in FIG. 10 B;
c. in a cross diagonally manner, where one electrode is near one vertebrate and the other electrode is near the second following vertebrate, as shown in FIG. 10 C;
d. in a cross diagonally manner, between said vertebrates, where one is close to said spine and the other is far from said spine, as shown in FIG. 10 D;
e. in a cross diagonally manner, between the vertebrates, as shown in FIG. 10 E;
f. in a cross diagonally manner, where one electrode is near one vertebrate and the other electrode is near the following vertebrate, as in FIG. 10 F;
g. in a cross diagonally manner, between said vertebrates, as shown in FIG. 10 G;
h. in a cross diagonally manner, between said vertebrates, where both said electrodes are on the same side of said spine, as shown in FIG. 10 H;
i. in a cross diagonally manner, where one electrode is near one vertebrate and the other electrode is far from the second following vertebrate, as shown in FIG. 10 I;
or any combination thereof.

It is another object of the present invention to provide another system [1] for controlling and modulating a mammalian's body temperature and thermoregulation system, comprising:
a. a display monitor [30];
b. a pulse generator [10];
c. at least one channel of electrical or electromagnetic stimuli configured by at least one pair of stimulating electrodes [5], connected to said pulse generator [10], and adapted for cutaneous application on said body's spinal cord, transcutaneously or by minimally invasive means;
d. at least one measurement unit for measuring physiological parameters, and
e. a control panel [20], configured for receiving, and analyzing said measured physiological signals, implementing and commanding an optimal pulse stimulating procedure to said pulse generator [10]; for controlling said pulse generator [10] and said at least one pair of stimulating electrodes [5].
   wherein said control panel [20] is adapted to implement said optimal pulse stimulating procedure for blocking the afferent neural pathways, evoking said afferent neural pathways, attenuating said afferent neural pathways, blocking and evoking said efferent neural pathways, blocking and attenuating said efferent neural pathways, attenuating and evoking, or blocking, attenuating, and evoking said efferent neural pathways, involved in said thermoregulation system, thereby controlling and modulating said body's temperature and thermoregulation system.

It is another object of the present invention to provide the other system [1] as defined above, wherein said a pulse generator [10] is selected from the group consisting of:
a. Trans Electrical Neuro Stimulation (TENS)
b. Interferential Current (IFC)
c. Electro Magnetic stimulation (EMS)
or any combination thereof.

It is another object of the present invention to provide the other system [1] as defined above, wherein said measurement unit is at least one unit selected from the group consisting of:
a. a temperature (T) unit [70], used for measuring skin and core temperature;
b. a Heart Rate Variability (HRV) unit [50], used for measuring heart beat;
c. an Electromyography (EMG) unit [60], used for measuring muscles electrical activity;
d. an oxygen utilization (Vo2) measuring unit [80], and
e. a neuro-signal unit [90], used for measuring neuro-electric activity;
or any combination thereof.

It is another object of the present invention to provide the other system [1] as defined above, wherein said control panel [20] is adapted to command at least one predetermined pulse stimulating procedure selected from the group consisting of:
stimulating the fast conduction Aβ fibers, thereby modulating the “thermal input” transferred to the hypothalamus;
stimulating said Aβ fibers for controlling the passage of sensory information traveling to said hypothalamus;
stimulating the slow conduction C fibers for a “heat” input, thereby modulating said thermal input received by said hypothalamus;
stimulating said C fibers for blocking said “heat” input, thereby modulating said thermal input received by said hypothalamus and avoiding new action potential to be generated;
stimulating the medium conduction Aδ fibers for a “cold” input message, thereby modulating said thermal input received by said hypothalamus;
stimulating said Aδ fibers for blocking the “cold” input message, thereby modulating said thermal input received by said hypothalamus and avoiding new action potential to be generated;
stimulation or blockage of efferent motor neurons receiving input from the thermoregulation system, related to changes in body temperature, thereby modulating said motor neurons activity;
stimulating the nerve fibers to simulate “cold” or “heat” input, from different parts of said body;
or any combination thereof.

It is another object of the present invention to provide the other system [1] as defined above, wherein said control panel [20] regulates said stimulating procedures of electric pulses by means of current's amplitude [A], frequency [F] and phase [φ], applied to said selected channels of electrical or electromagnetic stimuli.

It is another object of the present invention to provide the other system [1] as defined above, wherein said control panel [20] is configured to regulate said frequency [F] in the range of about 0 to about 30,000 [Hz].

It is another object of the present invention to provide the other system [1] as defined above, wherein said control panel [20] is configured to regulate said frequencies' [F] beat in the range of about 0 to about 1000 [Hz].

It is another object of the present invention to provide the other system [1] as defined above, wherein said control panel [20] is configured to implement said stimulating procedures in cycles which said frequencies' [F] beat are not constant between cycle to cycle.

It is another object of the present invention to provide the other system [1] as defined above, wherein said control panel [20] is configured to regulate said frequency [F] in a manner selected from the group consisting of: linear, nonlinear, sinusoidal, or any combination thereof.

It is another object of the present invention to provide the other system [1] as defined above, wherein said control panel [20] is configured to implement said stimulating procedures in intervals which range from about 0 to about 300 [sec].

It is another object of the present invention to provide the other system [1] as defined above, wherein said control panel [20] is adapted to implement said pulse stimulating procedure for blocking the temperature signals traveling from the entire body or selected body parts from reaching the brain, thereby isolating the thermoregulation system from reacting to changes in skin or core temperature

It is another object of the present invention to provide the other system [1] as defined above, wherein said control panel [20] is adapted to implement said stimulating procedure adapted to control and modulate said body's thermoregulation system to at least one of the following:
a. maintain normo-thermia or sensation of normo-thermia;
b. either induce or maintain hypo-thermia or sensation of hypo-thermia;
c. either induce or maintain hyper-thermia or sensation of hyper-thermia.

It is another object of the present invention to provide the other system [1] as defined above, wherein said control panel [20] is adapted to implement said stimulating procedure adapted to control and modulate said thermoregulation system physiological responses such as: vasodilatation, vasoconstriction of blood vessels, shivering or heart rate, by affecting the efferent neurological pathways.

It is another object of the present invention to provide the other system [1] as defined above, wherein said at least one pair of stimulating electrodes [5] are configured for predetermined location selected from the group consisting of: topically close to the spinal cord, remote from said spinal cord, transcutaneously, or by means of minimally invasive means to said spinal cord, or any combination thereof.

It is another object of the present invention to provide the other system [1] as defined above, wherein said at least one pair of stimulating electrodes [5] are configured to be attached to said body using at least one patch [6].

It is another object of the present invention to provide the other system [1] as defined above, wherein said at least one pair of stimulating electrodes [5] are further configured to operate as sensors for measuring Electro-Cardio-Graph (ECG) signals.

It is another object of the present invention to provide the other system [1] as defined above, wherein said least one pair of stimulating electrodes [5] are further configured to operate as sensors for measuring Neuro-Electric signals.

It is another object of the present invention to provide the other system [1] as defined above, wherein said control panel [20] is adapted to command said stimulation procedure along the whole length of the spinal cord or selected segments thereof, according to required treatment and clinical need.

It is another object of the present invention to provide the other system [1] as defined above, wherein said control panel [20] is adapted to command and implement said stimulation procedure for controlling said body's temperature and thermoregulation system for the entire body or selected body parts.

It is another object of the present invention to provide the other system [1] as defined above, wherein control panel [20] is adapted to command said stimulation procedure for simultaneously controlling said selected body parts, each for normo-thermia, hyper-thermia or hypo-thermia.

It is another object of the present invention to provide the other system [1] as defined above, wherein said system [1] further comprises means for the application of medication such as: local or general anesthetics, analgesics and or sedation agents, muscle blocking or relaxants agents, pain blocking agents, consciousness impairing agents.

It is another object of the present invention to provide the other system [1] as defined above, wherein said control panel [20] controls said application of stimulation procedure automatically.

It is another object of the present invention to provide the other system [1] as defined above, wherein said control panel [20] controls said application of stimulation procedures at least partially manually.

It is another object of the present invention to provide the other system [1] as defined above, wherein said display monitor [30] is configured to display parameters selected from the group consisting of: said measurement units measurements, said control panel [20] optional said stimulating procedures, said control panel [20] said administrated stimulating procedure, or any combination thereof.

It is another object of the present invention to provide the other system [1] as defined above, wherein said control panel [20] and said display monitor [30] are combined into a single device, such as a touch screen.

It is another object of the present invention to provide the other system [1] as defined above, wherein said control panel [20], said display monitor [30] and said pulse generator [10] are combined into a single device.

It is another object of the present invention to provide the other system [1] as defined above, wherein said system [1] further comprises at least one Heat Exchange Unit (HEU) [100] for heating or cooling said body.

It is another object of the present invention to provide the other system [1] as defined above, wherein said HEU [100] further comprises: at least one thermal sensor [101], an HEU analysis unit [102], an HEU activating unit [103] and at least one of the following:
an HEU output to heaters unit [109] and at least one heater [105]
an HEU output to coolers unit and at least one cooler.

It is another object of the present invention to provide the other system [1] as defined above, wherein said heaters [105] or said coolers are placed on a body part selected from the group consisting of: hand palms, feet soles, wrapped around large body parts, or any combination thereof.

It is still an object of the present invention to provide the other system [1] as defined above, wherein said least one pair of stimulating electrodes [5] are configured to be placed parallel to the vertebral column and over the intervertebral foramen, thereby enabling stimulation of the appropriate roots of spinal nerves which supply the affected dermatome.

It is lastly an object of the present invention to provide the other system [1] as defined above, wherein said least one pair of stimulating electrodes [5] layout in relation to the spinal cord vertebrates in a configuration selected from the group consisting of:
a. in a perpendicular manner, between said vertebrates as shown in FIG. 10 A;
b. in a perpendicular manner, across said vertebrates as shown in FIG. 10 B;
c. in a cross diagonally manner, where one electrode is near one vertebrate and the other electrode is near the second following vertebrate, as shown in FIG. 10 C;
d. in a cross diagonally manner, between said vertebrates, where one is close to said spine and the other is far from said spine, as shown in FIG. 10 D;
e. in a cross diagonally manner, between the vertebrates, as shown in FIG. 10 E;
f. in a cross diagonally manner, where one electrode is near one vertebrate and the other electrode is near the following vertebrate, as in FIG. 10 F;
g. in a cross diagonally manner, between said vertebrates, as shown in FIG. 10 G;
h. in a cross diagonally manner, between said vertebrates, where both said electrodes are on the same side of said spine, as shown in FIG. 10 H;
i. in a cross diagonally manner, where one electrode is near one vertebrate and the other electrode is far from the second following vertebrate, as shown in FIG. 10 I;
or any combination thereof.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which

FIG. 1: presents a simplified pictorial illustration of the system's [1] stimulating electrodes [5] placed on the spinal cord.

FIG. 2: presents an illustrated view of the human spine.

FIG. 3: presents a diagram for the system [1] and its component, including the optional MAC units.

FIG. 4: presents a diagram for the temperature control algorithm.

FIG. 5: presents a diagram for the Heart Rate Variability (HRV) unit [50].

FIG. 6: presents a diagram for the Electromyography (EMU) unit [60].

FIG. 7: presents a diagram for the Temperature measurement unit [70].

FIG. 8: presents a diagram for the oxygen utilization (Vo2) unit [80].

FIG. 9: presents a diagram for the Neuro-signal unit [90].

FIGS. 10a-10i: present examples regarding the electrodes layout in relation to the spinal cord vertebrate.

FIG. 11: presents a diagram for Heat Exchange Unit (HEU) [100].

FIG. 12: presents an illustrated current application to the spine.

FIG. 13: presents an illustrated electromagnetic field created by a solenoid.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a system and method for neuromodulation of body temperature regulation system.

In particular, the present invention relates to a system and method for neuromodulation of the body's temperature regulation system, for controlling, monitoring and attenuating the body's temperature and thermoregulation system and physiological responses, such as vasodilatation, vasoconstriction of blood vessels or shivering.

Current methods only affect the external input to the body's system without directly modulating the main internal “thermostat” system.

According to different embodiments of the present invention, the presented technical solution answers the need for controlling and monitoring the body's temperature and thermoregulation physiological responses, such as but not limited to, vasodilatation or vasoconstriction of blood vessels or shivering, and provides a system [1] and method for neuromodulation of the body temperature regulation system.

Reference is now made to a system [1] for controlling and modulating a mammalian's body temperature and thermoregulation system, comprising:
a. a display monitor [30];
b. a pulse generator [10];
c. at least one channel of electrical or electromagnetic stimuli configured by at least one pair of stimulating electrodes [5], connected to the pulse generator [10], and adapted for cutaneous application on the body's spinal cord, transcutaneously or by minimally invasive means;
d. a control panel [20], for controlling the pulse generator [10] and the at least one pair of stimulating electrodes [5], and
e. at least one Measurement Analysis and Command (MAC) unit for measuring physiological parameters, analyzing their signals and commanding an optimal stimulating procedure to the control panel [20];
   wherein the control panel [20] and the MAC units are adapted to implement the optimal stimulating procedure for blocking the afferent neural pathways, evoking the afferent neural pathways, attenuating the afferent neural pathways, blocking and evoking the efferent neural pathways, blocking and attenuating the efferent neural pathways, attenuating and evoking, or blocking, attenuating, and evoking the efferent neural pathways, involved in the thermoregulation system, thereby controlling and modulating the body's temperature and thermoregulation system.

The pulse generator [10] is selected from the group consisting of:
a. Trans Electrical Neuro Stimulation (TENS)
b. Interferential Current (IFC)
c. Electro Magnetic stimulation (EMS)
or any combination thereof.

The MAC unit is at least one unit selected from the group consisting of:
a. a temperature (T) unit [70], used for measuring skin temperature, core temperature, or skin and core temperature;
b. a Heart Rate Variability (HRV) unit [50], used for measuring heart beat;
c. an Electromyography (EMG) unit [60], used for measuring muscles electrical activity;
d. an oxygen utilization (Vo2) measuring unit [80], for measuring oxygen utilization and
e. a neuro-signal unit [90], used for measuring neuro-electric activity;
   or any combination thereof; the MAC units adapted to receive and analyze the measured signals and to command the administration of the optimal pulse stimulating procedure to the control panel [20].

Reference is now made to a method for controlling and modulating a mammalian's body temperature and thermoregulation system, the method comprising steps of
a. obtaining a system [1] for controlling and modulating a mammalian's body temperature and thermoregulation system, comprising:
   i. a display monitor [30];
   ii. a pulse generator [10];
   iii. at least one channel of electrical or electromagnetic stimuli configured by at least one pair of stimulating electrodes [5], connected to the pulse generator [10], and adapted for cutaneous application on the body's spinal cord, transcutaneously or by minimally invasive means;
   iv. a control panel [20], for controlling the pulse generator [10] and the at least one pair of stimulating electrodes [5], and
   v. at least one Measurement Analysis and Command (MAC) unit for measuring physiological parameters, analyzing their signals and commanding an optimal pulse stimulating procedure to the control panel [20];
b. placing the electrodes [5] over the skin bilaterally along the body's back;
c. measuring the physiological parameters;
d. receiving the measured physiological parameters;
e. analyzing the measured physiological parameters;
f. determining whether a change in the parasympathetic activity in the body requires activating the pulse generator [10], due to a change in the patient's body temperature;
g. determining which electrodes, placed on which dermatome, should be stimulated;
h. determining an optimal stimulation procedure;
i. stimulating the optimal stimulation procedure, at below pain threshold level, thereby inducing sense of heat or coldness;
j. stimulating the optimal stimulation procedure, for covering all somatic dermatomes, such that all incoming thermal data is subject to filtering or modulation, and
k. monitoring the measured physiological parameters as feedback signals for monitoring the applied stimulating procedure, and commanding the control panel [20] accordingly;
   wherein the optimal pulse stimulating procedure is adapted to block the afferent neural pathways, evoke the afferent neural pathways, attenuate the afferent neural pathways, block and evoke the efferent neural pathways, block and attenuate the efferent neural pathways, attenuate and evoke, or block, attenuate, and evoke the efferent neural pathways, involved in the thermoregulation system, thereby controlling and modulating the body's temperature and thermoregulation system. The method further comprises the step of analyzing the input signals from the HRV unit in time and in frequency domains;

Reference is now made to an alternative system [1] for controlling and modulating a mammalian's body temperature and thermoregulation system, comprising:
a. a display monitor [30];
b. a pulse generator [10];
c. at least one channel of electrical or electromagnetic stimuli configured by at least one pair of stimulating electrodes [5], connected to the pulse generator [10], and adapted for cutaneous application on the body's spinal cord, transcutaneously or by minimally invasive means;
d. at least one measurement unit for measuring physiological parameters, and
e. a control panel [20], configured for receiving, and analyzing the measured physiological signals, implementing and commanding an optimal pulse stimulating procedure to the pulse generator [10]; for controlling the pulse generator [10] and the at least one pair of stimulating electrodes [5].
   wherein the control panel [20] is adapted to implement the optimal pulse stimulating procedure for blocking the afferent neural pathways, evoking the afferent neural pathways, attenuating the afferent neural pathways, blocking and evoking the efferent neural pathways, blocking and attenuating the efferent neural pathways, attenuating and evoking, or blocking, attenuating, and evoking the efferent neural pathways, involved in the thermoregulation system, thereby controlling and modulating the body's temperature and thermoregulation system.

The measurement unit is selected from the group consisting of:
a. a temperature (T) unit [70], used for measuring skin and core temperature;
b. a Heart Rate Variability (HRV) unit [50], used for measuring heart beat;
c. an Electromyography (EMG) unit [60], used for measuring muscles electrical activity;
d. an oxygen utilization (Vo2) measuring unit [80], and
e. a neuro-signal unit [90], used for measuring neuro-electric activity;
or any combination thereof.

As described in details hereinbelow, the present invention provides a system [1] and method that may be used to achieve one or more of the following aims: maintain normo-thermia or sensation of normo-thermia, induce or maintain hypo-thermia or sensation of hypo-thermia, or induce or maintain hyper-thermia or sensation of hypo-thermia.

The physiological body temperature regulation system is a homeostatic system that attempts to maintain core body temperature within a certain limited range. It is a complex and dynamic system comprising a set of physiological responses that serve to increase or decrease core body temperature. It receives signals from sensors located both on the patient skin and also deep inside the body.

In the present invention, electrical or electromagnetic stimulation is used to filter the sensory information, e.g., pain, without wishing to be bound by theory, using “gate control”.

The sensory information travels through three types of fibers, but they all have different sub-classes to them. A, B, and C. A and B fibers are myelinated and C fibers are unmyelinated. A fibers are broken down further by conduction velocity into four main groups: α, β, γ and δ. In general, a chart of these fibers would look like this:
Aα, largest and fastest velocity, acts as motor and sensory fibers.
Aβ, next largest, acts as motor and sensory.
Aγ, next largest, acts as motor only.
Aδ, next largest, acts as sensory only.
B, smaller than A fibers, only acts a motor.
C, smallest, acts as motor and sensory.

In general, the A class of fibers are related to muscles (extrafusal and intrafusal fibers), Aδ sensory fibers relay touch, pressure, pain, and temperature, B motor fibers are for the autonomic system, while C sensory fibers are also for pain and temperature.

The thermal information, for “heat” or “cold”, travels through the same routes as the sensory information such as C fibers, Aδ fibers. By means of the “gate” principle, stimulation of the Aβ fibers, which has fast conduction, overcome other inputs, namely C fibers Aδ fibers, and thus attenuates the thermal information.

The information travels through the peripheral nerve system via the Aδ and C fibers to the spinal cord into the temperature regulation area in the brain, the hypothalamus. The hypothalamus processes the information and reacts accordingly. A series of temperature regulation mechanisms are activated to compensate for the loss or gain of core body temperature.

It has now been unexpectedly found that temperature signals passing through neural pathways between the periphery and the centrally-located temperature regulation centers, such as in the hypothalamus, may be blocked or modified by the cutaneous application of electrical pulses, similar to those used to block pain signals in Transcutaneous Electrical Nerve Stimulation (TENS) systems and its subset InterFerential currents (IFC) systems. Thus, in one aspect of the invention, a modified TENS system is used to block the temperature signals from reaching the brain. The system [1] isolates the hypothalamus from the information traveled via skin and core temperature sensors.

The electrical stimulation covers all somatic dermatomes, such that all incoming thermal data is subject to filtering. Using this technology enables the blocking or modulating the thermal input from reaching the hypothalamus. Thereby body's temperature management could be performed in a safer manner, more efficient and easier by isolating the body's “thermostat” from reacting to active cooling of the body.

The system [1] of the present invention thereby facilitates efficient temperature manipulation of either the entire body or regions thereof for in routine clinical setting, with or without general anesthesia.

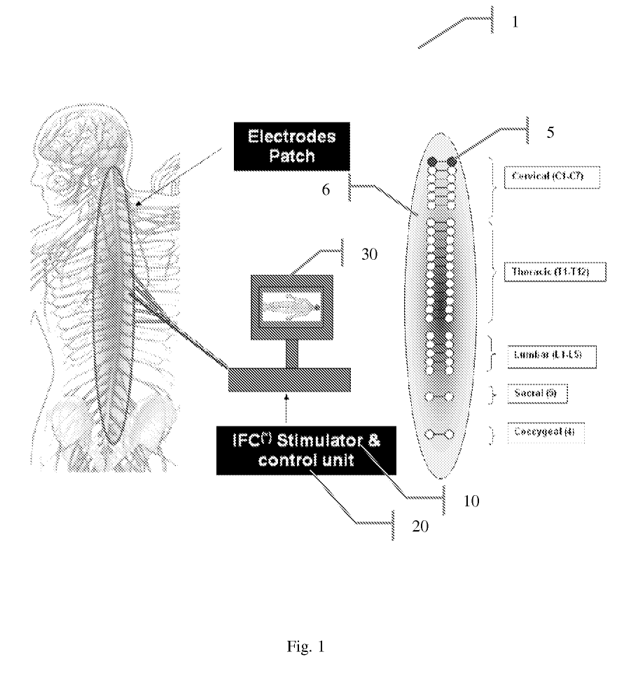

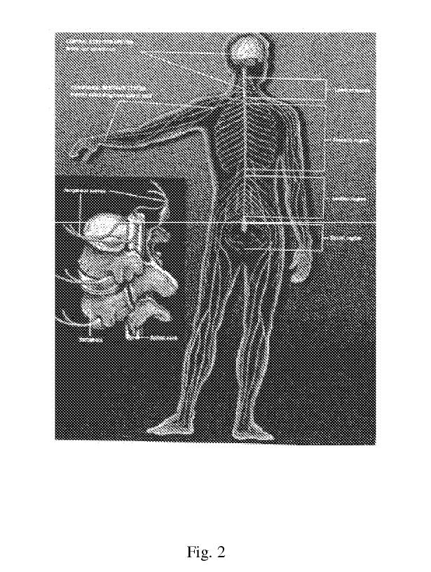

In humans, as represented in FIGS. 1 and 2, thirty-one pairs of spinal nerves emerge from the vertebral column via the intervertebral foramen. Each spinal nerve is formed by the union of ventral (motor) and dorsal (sensory) roots which unite in the intervertebral foramen to form a mixed spinal nerve. Placement of the electrodes [5] parallel to the vertebral column (para-spinal application) and over the intervertebral foramen permits the stimulation of the appropriate roots of spinal nerves which supply the affected dermatome, i.e. the area of skin which receives its nerve supply from a specified spinal nerve. Thermal input that is generated from the different body areas and travels through these nerves are blocked or modulated using the system [1] of the present invention.

The present invention primary provides a system [1] and method for controlling and modulating the body's temperature in a mammalian, preferably human subject, by means of applying electrical or electromagnetic stimulation in proximity to peripheral nerves that play a role in the thermoregulation system. Electrical stimulation can be generated by electrical pulses and electromagnetic stimulation for example by placing solenoids (FIG. 13) along the spinal cord.

FIG. 1 presents an illustrated set-up of the system [1] components in the clinical settings. A patch [6] of electrode array [5] is placed on the back of the patient covering both sides of the spinal cord. The patch [6] is connected to a stimulator [10] (can be IFC or electro-magnetic stimulator) that control the pulses that are generated.

The method of the present invention comprises applying electrical or electromagnetic stimuli, variable in: intensity, frequency, amplitude, waveform, wave length, pulse intervals length and phase, such as bi-phasic either in proximity to the body, topically, over the skin; either close to the spinal cord or remote therefrom, transcutaneously or by minimally invasive means to the spinal cord via electrode pads [5]. The electrical pulses, variable in signal intensity, frequency, amplitude, waveform, wavelength, pulse interval length and phase, manipulate the temperature signals that travel from the entire body or selected body parts to and from the brain to isolate the thermoregulation system from reacting to changes in skin or core temperature.

The method of the present invention may be used in a number of different ways in order to achieve the desired thermoregulatory effect. These various different modes of operation (as will be described in the following section) may be grouped into the following three general classes of method:
1) Modulation of afferent thermal signals traveling to the hypothalamus;
2) Simulation of afferent signals by providing stimulatory pulses that are recognized by the hypothalamus as thermal signals; and
3) Modulation of efferent signals i.e. modulation of peripheral responses such as shivering and vasoconstriction that are initiated in response to hypothalamic activity.

The present invention includes within its' scope many different variants of these basic approaches. Some of these are listed below:

Stimulating Aβ Fibers:

Stimulating the fast conduction fibers Aβ to modulate the thermal input transferred to the hypothalamus.

Stimulating Aβ Fibers+C Fibers (“Heat” Simulation):

Stimulating the last conduction fibers Aβ and the slow conduction C fibers for “heat” input to modulate the thermal input received by the hypothalamus.

Stimulating Aβ Fibers+C Fibers (“Heat” Simulation)+Aδ (“Cold” Input Blockage):

Stimulating the fast conduction fibers Aβ, the slow conduction C fibers for “heat” input and blocking the medium conduction Aδ fibers input to modulate the thermal input received by the hypothalamus.

Stimulating Aβ Fibers+Aδ (“Cold” Stimulation):

Stimulating the fast conduction fibers Aβ and the medium conduction Aδ fibers for “cold” input to modulate the thermal input received by the hypothalamus.

Stimulating Aβ Fibers+Aδ (“Cold” Stimulation)+C Fibers (“Heat” Input Blockage):

Stimulating the fast conduction fibers Aβ, the medium conduction Aδ fibers to “cold” input and blocking slow conduction C fibers input to modulate the thermal input received by the hypothalamus.

Stimulating or Blockage of C Fibers Only:

Stimulate to modulate slow conduction C fibers input to modulate the thermal input received by the hypothalamus.

Stimulating or Blockage of Aδ Fibers Only:

Stimulate to modulate medium conduction Aδ fibers input to modulate the thermal input received by the hypothalamus.

Stimulating or Blockage of Motor Neurons:

Stimulate to modulate motor neurons activity due to input received from the hypothalamus related to changes in body temperature.

Partial Body Coverage (“Cold” Areas and/or “Hot” Areas Simultaneously):

Stimulating the nerve fibers to simulate “cold” or “heat” input from different parts of the body.

Thus, in one embodiment, the method comprises the application of a variety of electrical pulses, variable in signal intensity, magnitude and phase that simulate thermal information (“heat” or “cold”) from the entire body or selected body parts to manipulate the body thermoregulation system.

Without wishing to be bound by theory, the body reacts to the stimulating pulses as if it was cooled or heated without the actual application of “heat” or “cold”.

The method optionally comprises applying a variety of electrical pulses and intensities directly to the C fibers or Aδ fibers to manipulate or block sensory information passage through these roots.

In a further embodiment, the method comprises applying a variety of electrical or electromagnetic stimuli variable in: intensity, frequency, amplitude, waveform, wave length, pulse intervals length and phase, such as bi-phasic, wherein the stimuli is used to block afferent thermoregulatory signals that have their origin in the periphery and would otherwise travel to the hypothalamic temperature regulation centers.

In this embodiment, the system [1] can block information from traveling on C fibers and Aδ fibers by stimulating these nerves by pulses to maintain constant refractory period and avoiding new action potential to be generated. The system [1] stimulates the Aβ fibers to control passage of sensory information traveling to the brain. The current delivered to each pair of electrodes [5] can be optimized according to the degree of current travelling through the body to the next pair.

In another aspect, the method of the invention comprises applying a variety of electrical pulses, variable in signal intensity, magnitude and phase to the efferent motor neurons receiving input from the thermoregulation system to manipulate or block vasodilatation or vasoconstriction of blood vessels or shivering.

The stimulating procedures of electric pulses are regulated by means of current's amplitude [A], frequency spectrum [F] and phase [φ], applied to selected electrode pairs [5], where signals are variable in: intensity, frequency amplitude, waveform, wave length, pulse intervals length and phase, such as bi-phasic.

In reference to the system [1] of the present invention, in one embodiment, the system [1] comprises a multitude of electrode pair pads [5] connected to a pulse generator [10] for electrical stimulation, as shown in FIG. 2.

An example of suitable off-the-shelf Pulse Generator is the ANALGESIC PULSER EMS PROFESSIONAL DIGITAL DELUXE EMS MACHINE, Model: AP-439, supplied by BUYAMAG INC. CA, USA. An example of suitable off-the-shelf electrodes are the DURA-STICK® II Self-adhesive Electrodes 2″ Square (40) (Model: 42041) manufactured by the Chattanooga Group, Hixson Tenn., USA.

The system [1] further comprises a control panel [20] and a display monitor [30], both connected to the pulse generator [10].

The pulse generator [10] generates electrical pulses variable in signal intensity, magnitude and phase, according to the particular clinical needs i.e. partial or entire body cooling or heating. In one preferred embodiment, the system [1] comprises a set of 31 pairs of percutaneous electrodes [5] attached by stickers onto the dorsal skin of the patient on both sides of the spinal cord. The stimulation can be applied along the whole length of the spinal cord or applied only selected segments (selected number of electrode pairs [5]) thereof, depending on the required treatment and clinical need.

The type of pulse generated (intensity, magnitude, phase and waveform) is optionally controlled by the operator via operator-generated input through control panel [20]. In this way, the desired pulse characteristics required in order to block the body thermal information from reaching the brain, or to block the efferent passage thermal information from the brain along neuromotor pathways, may be determined and controlled.

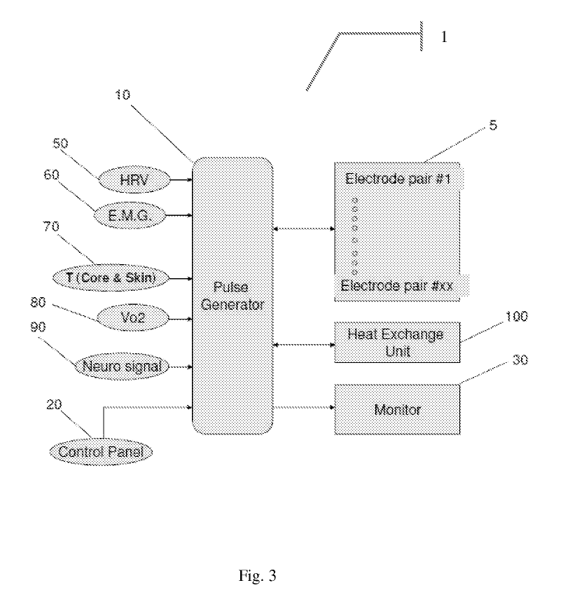

According to a preferred embodiment of the present invention, the system [1] comprises at least one Measurement Analysis and Command (MAC) unit that receive various types of physiological signals from the body, as shown in FIG. 3. The measured signals are used to determine whether the pulse generator [10] should be activated, and the type of pulse procedure to be generated. The MAC units are further adapted to analyze the measured physiological signals as feedback signals for monitoring the applied pulse stimulating procedures, and command the control panel [20] accordingly.

The system [1], according to this preferred embodiment, comprises one or more of the following Measurement Analysis and Command (MAC) units:
A. A Heart Rate Variability (HRV) unit [50];
B. an Electromyography (E.M.G.) unit [60];
C. a temperature measuring T(skin and core) unit [70];
D. an oxygen utilization [Vo2] measuring unit [80];
E. a neuro-signal unit [90];

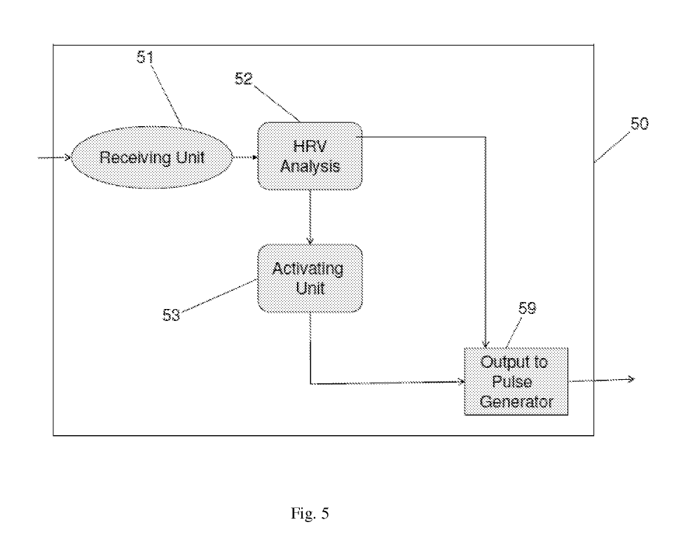

A. The Heart Rate Variability (HRV) unit [50], shown in FIG. 5, comprises a receiving unit [51] that receives Electro-Cardio-Gram (ECG) signals from an off-the-shelf device that measures HRV activity in the body.

An example of a suitable off-the-shelf measuring device is the model—Heart Rhythm Scanner 2.0, manufactured by Biocom Technologies, Poulsbo Wash., USA, or the model PageWriter TC50 cardiograph, manufactured by Koninklijke Philips Electronics N.V.

In one preferred embodiment, the receiving unit [51] receives electrocardiogram signals from a separate set of electro cardio graph sensors or from the present invention system [1] electrode pads [5] that not only function as electric pulse stimulators but also additionally function as Electro-Cardio-Graph sensors that measure and transfer the electrocardiogram data signals from the body to the HRV receiving unit [51].

The data is then transferred to the HRV analysis unit [52]. The HRV analysis unit [52] analyses, according to a dedicated application, the received signals that indicate parasympathetic activity in the body. The signals are analyzed in time and frequency domains. The HRV analysis unit [52] determines whether a change in the parasympathetic activity in the body requires activating the pulse generator [10] due to a change (or an expected change) in the patient's body temperature (thus requiring the stimulating pulses) or whether the parasympathetic activity in the body is “normal” and thus there is no need to activate pulse generator [10]. The HRV analysis unit [52] also determines the optimal blocking pulse to be generated i.e. optimal intensity, optimal magnitude and optimal phase. This is determined according to the electrocardiogram data received from the sensors.

If the parasympathetic activity in the body is “normal” the analysis unit [52] transfers a signal to the “output to Pulse Generator” unit [59] indicating that the system [1] should stay as is, i.e. the pulse generator [10] should be activated in its default state.

If the parasympathetic activity in the body requires activating the pulse generator [10] then an activating signal is transferred to the activating unit [53]. The activating unit [53] then sends a signal to the “output to Pulse Generator” unit [59] indicating an activation request including the optimal blocking pulse characteristics. The data from the “output to Pulse Generator” unit [59] is transferred out of the HRV unit [50] and into the pulse generator [10].

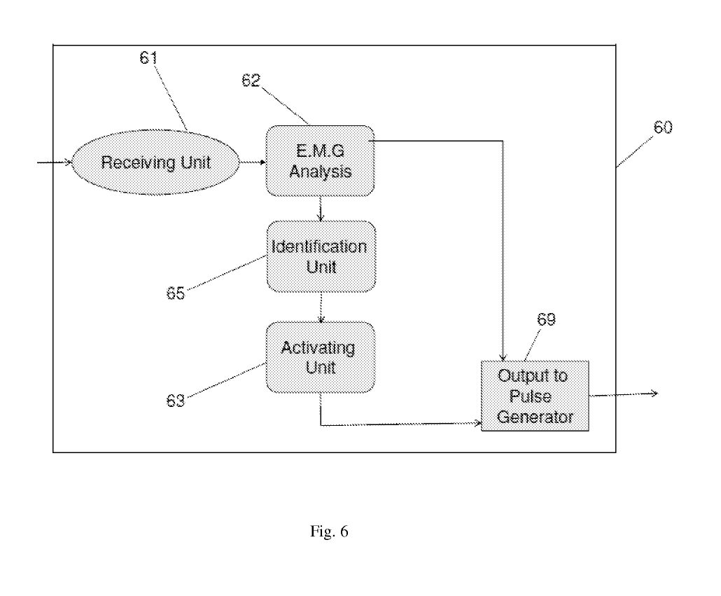

B. The Electromyography (E.M.G.) unit [60], shown in FIG. 6, comprises a receiving unit [61] that receives E.M.G. data signals from an off-the-shelf device that measures E.M.G. activity in the body by appropriate sensors placed on the body.

An example of a suitable off-the-shelf measuring device is the model—Neuropack M1 manufactured by NIHON KOHDEN CORPORATION, JAPAN.

The data signals are then transferred to the EMU analysis unit [62]. The EMG analysis unit [62] analyses, according to a dedicated application, the received signals that indicate the EMG activity in the body. The EMG analysis unit [62] determines whether the EMG activity in the body is representative of shivering or muscle movement, thus requiring activation of the pulse generator [10] due to a change, or an expected change, in the patient's body temperature and thus requiring the blocking pulses or whether the EMG activity in the body is “normal”, when shivering does not occur, and thus there is no need to change the pulse generator [10] activity.

If shivering does not occur, EMG analysis unit [62] transfers a signal to the “output to pulse generator” unit [69] indicating that the system [1] should stay as is, i.e. the pulse generator [10] should be activated in its default state.

If shivering occurs, then the EMG data is transferred to the dermatome identification unit [65]. The dermatome identification unit [65] analyzes the EMG data received by means of a dedicated application, and determines which dermatomes are affected by the EMG muscle movement (i.e. shivering) data. Then the identification unit [65] also determines, by means of a dedicated application, the optimal blocking pulse to be generated i.e. optimal intensity, optimal magnitude and optimal phase. Identification unit [65] also determines to which electrodes [5], placed on which dermatome, the pulses should be generated hereinafter referred to as the target electrodes [5]. This is determined according to the electro-cardio-gram data received from the sensors, by means of a dedicated application.

The information concerning the optimal blocking pulses and the target electrodes [5] is transferred to the activating unit [63]. The activating unit [63] then sends a signal to the “output to pulse generator” unit [69] indicating an activation request including the optimal blocking pulses and the target electrodes [5]. The data from the “output to Pulse Generator” unit [69] is transferred out of the E.M.G. unit [60] and into the pulse generator [10].

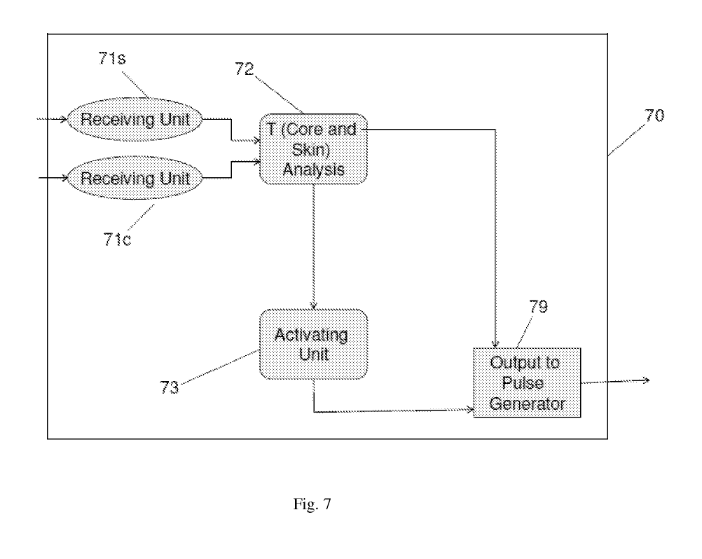

C. The temperature measuring T(skin and/or core) unit [70], shown in FIG. 7, comprises a receiving unit [71c] that receives the body core temperature data from an off-the-shelf thermal measuring device. The core temperature data received from the thermal measuring device is measured by means of thermal sensors placed in the rectum, or the bladder, or the tympanic membrane or the esophagus, or any combination thereof.

The temperature measuring T(skin and/or core) unit [70] further comprises a receiving unit [71s] that receives the body's skin or core temperature data from a second off-the-shelf thermal measuring device. The skin or core temperature data received from the thermal measuring device is measured by means of thermal sensors placed on certain locations on the body skin as determined by the operator.

The data from [71c] and [71s] is then transferred to the T(skin and/or core) analysis unit [72]. The analysis unit [72] analyses, according to a dedicated application, the received temperature data. If the body temperature changes in a rate faster or slower than a predetermined set point rate, then T(skin and/or core) analysis unit [72] will choose to activate the pulse generator [10]. The set point rate is determined and set by the operator via control panel [20] also connected to T(skin and/or core) analysis unit [72] (not shown).

For example, If the body core heat rises at an average rate slower than 0.1° C. every 3 minutes (2° C. an hour), then the pulse generator [10] will be activated to cool the body. The T(skin and/or core) analysis unit [72] also determines the optimal blocking pulse to be generated i.e. optimal intensity, optimal magnitude and optimal phase. This is determined according to the temperature data received from the thermal measuring devices. This determination is achieved by means of a dedicated application.

If the temperature changing rate is faster than the pre set rate T(skin and/or core) analysis unit [72] transfers a signal to the “output to Pulse Generator” unit [79] indicating that the system [1] should stay as is i.e. the pulse generator [10] should not change its activity.

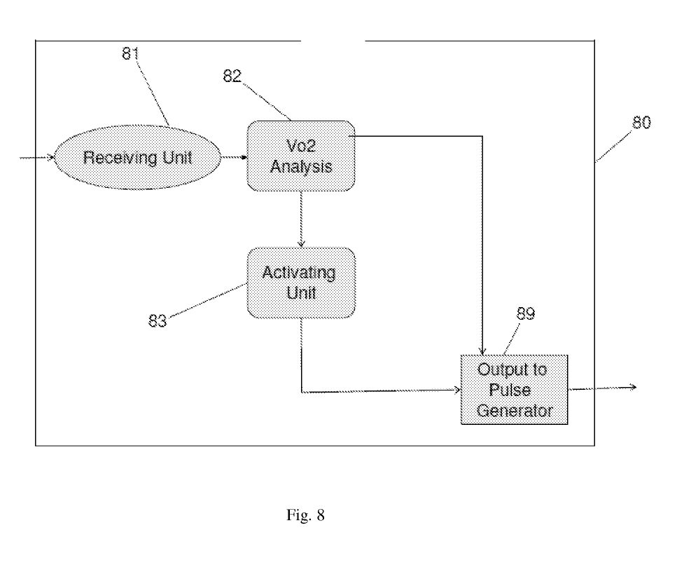

D. The breathing measuring [Vo2] unit [80], shown in FIG. 8, comprises a receiving unit [81] that receives breathing oxygen rate signals from an off-the-shelf device that measures the breathing oxygen rate of the body.

An example of a suitable off-the-shelf measuring device is the model—ECG-VO2—the CPX-system, manufactured by Cortex Biophysik GmbH, Germany.

The data is then transferred to the Vo2 analysis unit [82]. The Vo2 analysis unit [82] analyses, according to a dedicated application, the received signals that indicate the breathing oxygen rate of the body. The Vo2 analysis unit [82] determines whether the breathing activity in the body requires activating the pulse generator [10] due to a change, or an expected change, in the patient's body temperature, thus requiring the blocking pulses, or whether the breathing activity in the body is “normal” and thus there is no need to activate pulse generator [10].

For example, if the body breathing oxygen rate rises above 25% than a predetermined rate, then the pulse generator [10] will be activated. The Vo2 analysis unit [82] also determines the optimal blocking pulse to be generated i.e. optimal intensity, optimal magnitude and optimal phase. This is determined according to the breathing rate data received by the breathing rate device. This determination is achieved by means of a dedicated application.

If the breathing activity in the body is “normal” the analysis unit [82] transfers a signal to the “output to pulse generator” unit [89] indicating that the system [1] should stay as is i.e. the pulse generator [10] should not change its activity.

If the breathing activity in the body requires activating the pulse generator [10] then an activating signal is transferred to the activating unit [83] including the optimal blocking pulse. The activating unit [83] then sends a signal to the “output to Pulse Generator” unit [89] indicating an activation request including the optimal blocking pulse characteristics. The data from the “output to pulse generator” unit [89] is transferred out of the Vo2 unit [80] and into the pulse generator [10].

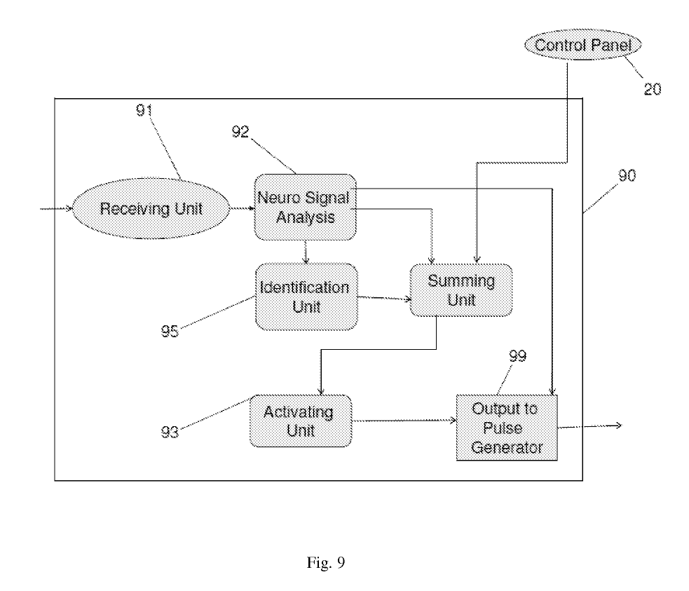

E. The neuro-signal unit [90], shown in FIG. 9, comprises a receiving unit [91] that receives electric-neuro-signals from an off-the-shelf device that measures neuro-electric activity in the body by placing appropriate sensors on the body. In one preferred embodiment, the receiving unit [91] receives neuro-electric signals from the present invention system [1] electrode pads [5] that not only function as electric pulse stimulators but also additionally function as neuro-electric sensors that measure and transfer the neuro-electric data signals from the body to the Neuro-signal receiving unit [91].

The data signals are then transferred to the Neuro-signal analysis unit [92]. The Neuro-signal analysis unit [92] analyses, according to a dedicated application, the received signals that indicate the neuro-electric activity in the body. The neuro Signal analysis unit [92] determines whether the neuro-electric activity in the body requires activating the pulse generator [10] due to a change, or an expected change, in the patient's body temperature and thus requiring the blocking pulses, or whether the neuro electric activity in the body is “normal” and thus there is no need to activate pulse generator [10].

If the neuro-electric activity in the body is “normal”, i.e., according to the set point, neuro-signal analysis unit [92] transfers a signal to the “output to pulse generator” unit [99] indicating that the system [1] should stay as is.

If the neuro-electric activity is not “normal”, then the neuro-electric data is transferred to the dermatome identification unit [95]. The dermatome identification unit [95] analyzes the neuro-electric data received by means of a dedicated application, and determines which dermatomes are affected by the neuro-electric data. Dermatome identification unit [95] also determines to which electrodes [5], placed on which dermatome, the pulses should be generated hereinafter referred to as the target electrodes [5]. This is determined according to the neuro-electric data received from the sensors, by means of a dedicated application.

The Neuro signal unit [92] also determines, by means of a dedicated application, the optimal blocking (opposite signal) pulse to be generated, i.e. optimal intensity, optimal magnitude and optimal phase, according to the received input signals.

Optionally, the operator can choose, via control panel [20] further connected to neuro-signal unit [90], the heat or cold characteristics of the desired pulse, i.e. whether to send “heat” or “cool” signals to the hypothalamus.

It is also optional for the operator to choose, via control panel [20], the appropriate dermatome instead of the dermatome identification unit [95].

The information concerning:
a. the optimal blocking pulses (from the Neuro-signal unit [92]),
b. the “cool”/“heat” characteristics (from the Neuro-signal unit [92] or from the control panel [20]) and
c. the target electrodes [5] (from the dermatome identification unit [95] or from the control panel [20]) is transferred to the summing unit [96] which sums the data in to one signal. The summed up signal (comprising all characteristics) is transferred to the activating unit [93]. The activating unit [93] then sends a signal to the “output to pulse generator” unit [99] indicating an activation request including the optimal blocking pulses, the “cool” or “heat” characteristics and the target electrodes [5]. The data from the “output to pulse generator” unit [99] is transferred out of the neuro signal unit [90] and into the pulse generator [10].

The pulse generator [10] receives the activation information, the optimal signals and the target electrodes [5], in the case of units [90] and [60], from the input units, and generates electric pulses accordingly.

The system [1] can also function using only one of the input units. The system [1] can also function without any of the input units by means of the operator inputting the desired pulse generating characteristics via control panel [20].

It should be understood that according to an embodiment of the present invention two or more of the system [1] input units could be applied and activated simultaneously.

If two or more of the input units are activated simultaneously, then the blocking pulses will be generated by the pulse generator [10] consequent upon the first input unit that requires a change in the generation of pulses according to the first input unit-output characteristics.

The system [1] can function automatically i.e. the electric pulses are generated by the pulse generator [10] automatically according to the output data of the input units.

The system [1] can also function be operated manually via a system [1] operator that receives all the data from the activated input units and views all the data on monitor [30]. The system [1] operator can determine according to the data viewed what type of pulses should be generated and generate them using control panel [20].

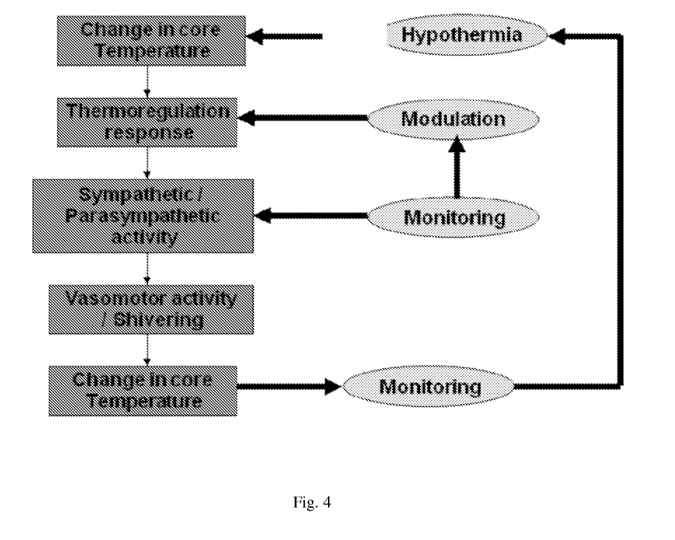

FIG. 4 as shown presents the bio feedback mechanism implemented in the in the invention (right side of the diagram). The left side of the flow-chart presents the cascade of events that takes place following a change in the body core temperature. Current methods monitor only the outcome of these events (temperature change) and induce “heat” or “cold”. The present invention monitors these events in an earlier stage, modulating directly the thermoregulation response, avoiding or delaying the triggering of vasomotor activity for better control on body temperature regulation.

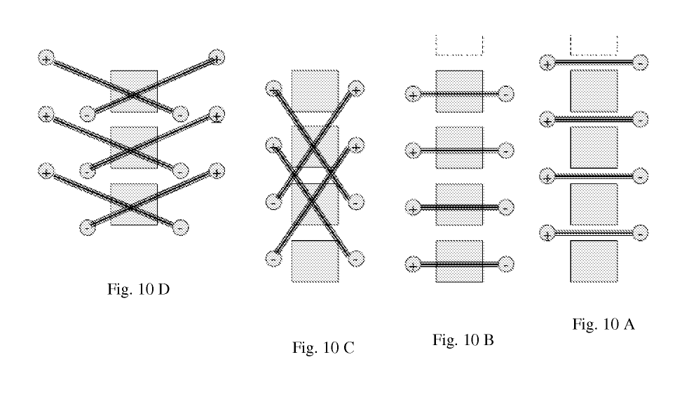

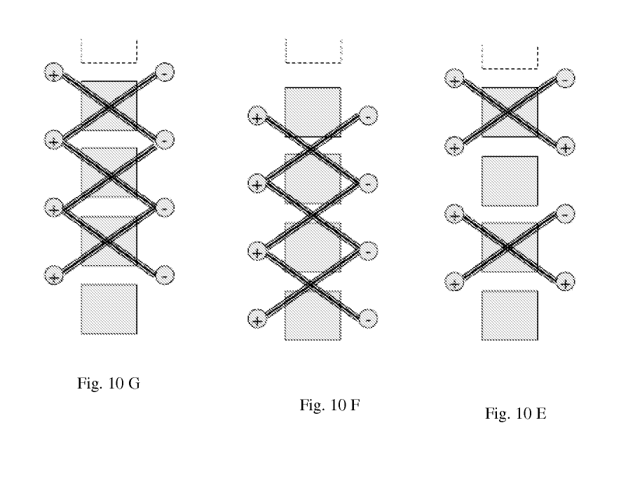

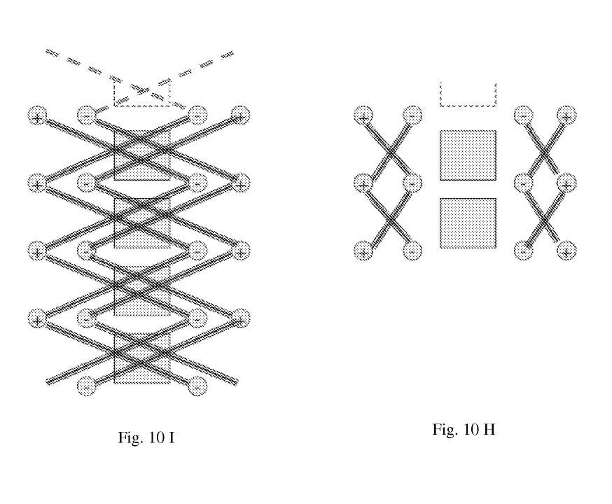

FIGS. 10A-10I present examples of several options, regarding the electrodes [5] layout for the electrical or electromagnetic stimuli, in relation to the spinal cord vertebrates. The electrodes [5] are applied by placing each pair of electrodes [5] in a manner such that the electrical current between the electrodes [5] flows in a configuration selected from the group consisting of:
a. in a perpendicular manner, between the vertebrate, as shown in FIG. 10 A;
b. in a perpendicular manner, across the vertebrate, as shown in FIG. 10 B;
c. in a cross diagonally manner, where one electrode is near one vertebrate and the other electrode is near the second following vertebrate, as shown in FIG. 10 C;
d. in a cross diagonally manner, between the vertebrate, where one electrode is close to the spine and the other electrode is far from the spine, as shown in FIG. 10 D;
e. in a cross diagonally manner, between the vertebrate, as shown in FIG. 10 E;
f. in a cross diagonally manner, where one electrode is near one vertebra and the other electrode is near the following vertebra, as in FIG. 10 F;
g. in a cross diagonally manner, between the vertebrate, as shown in FIG. 10 G;
h. in a cross diagonally manner, between the vertebrate, where both electrodes are on the same side of the spine, as shown in FIG. 10 H;
i. in a cross diagonally manner, where one electrode is near one vertebra and the other electrode is far from the second following vertebra, as shown in FIG. 10 I;
or any combination thereof.

The electrodes are placed in a manner according to the system [1] operator's decisions not limited to the layout manners shown in the figures herein, which serve only as examples.

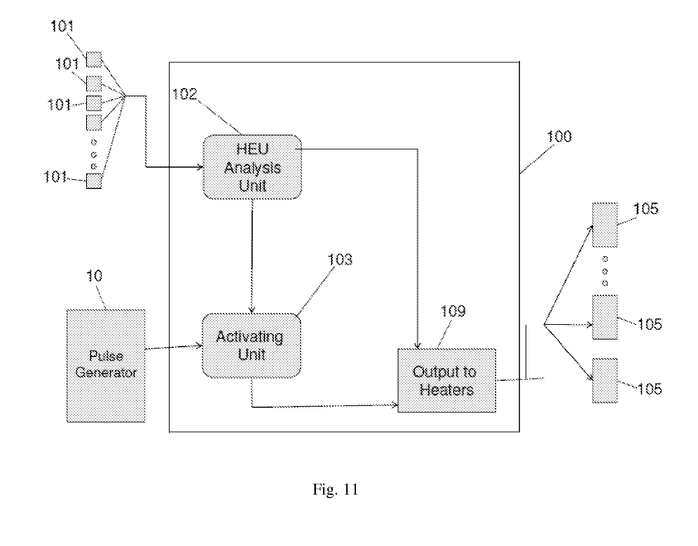

In one embodiment of the present invention the system [1] further comprises a Heat Exchange Unit (HEU) [100], shown in FIG. 11. The HEU [100] assists in heating the body and is applied in addition to the electric stimulation blocking pulses applied on the patient. The HEU comprises an analyzing unit [102] which receives the body skin or core temperature data measured by means of thermal sensors [101] placed on certain locations on the body skin or core according to the desire of the operator.

The HEU analysis unit [102] analyses, according to a dedicated application, the received signals that indicate the temperature of the body.

If the body temperature is “normal” the analysis unit [102] transfers a signal to the “output to heaters” unit [109] indicating that the heaters [105] should stay off.

If the body temperature exceeds a predetermined temperature, determined by the operator via control panel [20], then an activating signal is transferred to the activating unit [103]. The activating unit [103] then sends a signal to the “output to heaters” unit [109] indicating an activation request. The data from the “output to heaters” unit [109] is transferred out of the HEU [100] turning ON the heaters [105].

If further temperature signals are received indicating of the cooling of the body below the preset temperature, then a signal is sent to the activating unit [103] which sends a signal to the “output to heaters” unit [109] indicating a “stop activation” request, which is transferred to heaters [105] turning them off.

According to an embodiment of the present invention the HEU [100] can function only when appropriate signals are received from sensors [101] or it can also function automatically when the pulse generator [10] begins to generate blocking pulses, in which case an activating signal is transferred from the pulse generator [10] to HEU [100].

The heaters [105] are preferably placed on the hand palms and feet soles. As known in the art, these areas have the highest rate of sensors to skin surface, so heating these areas of the body is the most efficient. It should be noted that a similar HEU cooling unit can also be applied for cooling options in addition to the heaters, in a similar manner as described hereinabove.

Various different type of heat exchange module may be used in this embodiment of the system [1] of the present invention, including but not limited to, those based on Peltier-effect thermoelectric heat exchanger technology, computerized heating/cooling elements, elements wrapped around large body parts, as well as other suitable devices.

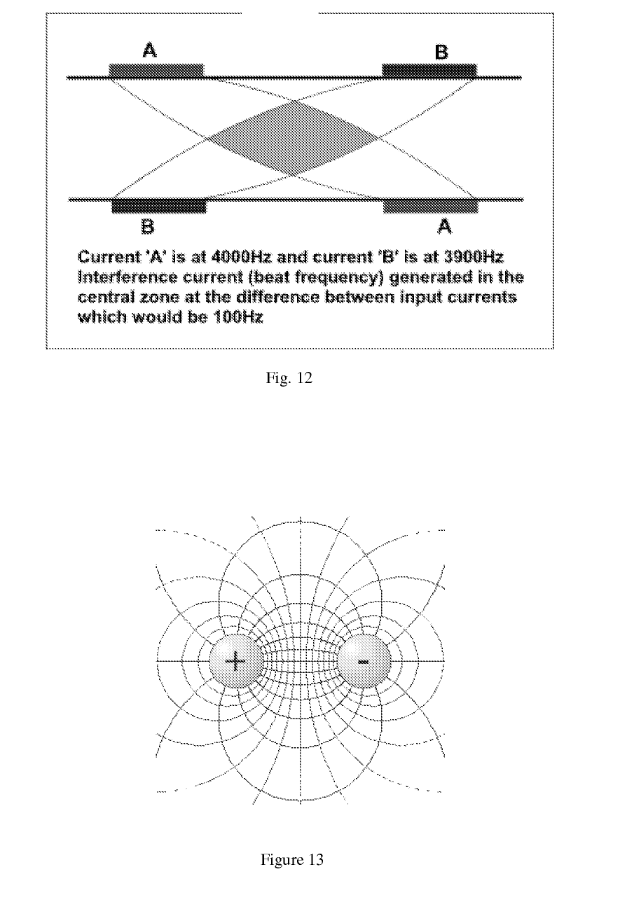

FIG. 12 as shown presents an example of the use of IFC technology to induce dipper body neuro-stimulation. The central zone with the requested frequency is generated dipper than skin level, allowing the stimulation of neural pathways that are located in the dipper layers. It also prevents from skin irritation that is resulted from the stimuli.

Examples for stimulating procedures and the stimulation regimes include:
1. Current A: 4,000 Hz
   Current B: 4,100 Hz
   Beat Frequency: 100 Hz. Stable.
   Interval: 5 sec stimulation, 5 sec no stimulation
2. Current A: 10,000 Hz
   Current B: 10,080 Hz
   Beat Frequency: 80 Hz. Stable.
   Interval: 0 sec
3. Current A: 2500 Hz
   Current B: 2630 Hz
   Beat Frequency: 80 to 130 Hz. Linear increase over 3 sec and decrease over 3 sec.
   Interval: 6 sec stimulation, 6 sec no stimulation
4. Current A: 2500 Hz
   Current B: 2600 Hz
   Beat Frequency: 100 Hz. Stable.
   Interval: 6 sec stimulation, 6 sec no stimulation
   Cycles: 100
   Followed by:
   Current A: 2500 Hz
   Current B: 2505 Hz
   Beat Frequency: 5 Hz. Linear increase over 3 sec and decrease over 3 sec.
   Interval: 6 sec stimulation, 6 sec no stimulation
   Cycles: 100

The range of currents (A, B) frequencies range is from about 0 to about 30,000 Hz

The beat frequency, i.e. the difference between the frequencies (A, B), range between about 0 to about 1000 Hz.

The change in frequency over time can be linear, nonlinear, sinusoidal, or any combination thereof.

The simulating procedure intervals may range from about 0 to about 300 [sec].

FIG. 13 as shown presents a demonstration of the electromagnetic field generated by a solenoid, without the need for a direct contact with the skin of the body.

The present invention technology includes:
Deeper stimulation at the desired frequency
Less power
Eliminate skin irritations
The application of constant low frequency, 1-4 Hz, with bursts of high frequency of 100 Hz.

The advantages of the present invention include, but not limited to:
Decreased temperature sensing
Regulation processes for fast induction; stable temperature maintenance and controlled re-warming.
Non-invasive methods.
Minimal use of medication therefore reducing costly side-effects.
Reducing stress associated with cold temperature side effects
Less cooling surface is needed
Non-invasive method with the effectiveness of an invasive one.
Can be used in non hospital settings
Can be used during transportation (EMS settings).
Can be used without the cumbersome heat exchange technology (no air/water/ice) as in Paltier technology.

It will be appreciated by a person skilled in the art that the present invention is not limited by what has been particularly shown and described hereinabove. Rather the scope of the present invention includes both combinations and sub-combinations of the features described hereinabove as well as modifications and variations thereof which would occur to a person of skill in the art upon reading the foregoing description and which are not in the prior art.

1-108. (canceled)

109. A system [1] configured to control and modulate a mammalian's body temperature and thermoregulation system, comprising:
a. a display monitor [30];
b. a pulse generator [10];
c. at least one channel of electrical and/or electromagnetic stimuli having at least one pair of stimulating electrodes [5], connected to said pulse generator [10], and configured for cutaneous application on said body's spinal cord, transcutaneously and/or by minimally invasive means;
d. a control panel [20], configured to control said pulse generator [10] and said at least one pair of stimulating electrodes [5]; and
e. at least one Measurement Analysis and Command (MAC) unit, configured to measure physiological parameters, analyze their signals and command an optimal stimulating procedure to said control panel [20];
wherein said control panel [20] and said at least one MAC unit are configured to implement said optimal stimulating procedure to afferent neural pathways involved in said thermoregulation system, such that said body's temperature and thermoregulation system are controlled and modulated.

110. The system [1] according to claim 109, wherein at least one of the following holds true:
a. said optimal stimulating procedure is selected form a group consisting of:
i) blocking said afferent neural pathways,
ii) evoking said afferent neural pathways,
iii) attenuating said afferent neural pathways, and
iv) any combination thereof;
b. said a pulse generator [10] is selected from a group consisting of:
i) Trans Electrical Neuro Stimulation (TENS),
ii) Interferential Current (IFC),
iii) Electro Magnetic stimulation (EMS), and
iv) any combination thereof;
c. said at least one MAC unit configured to receive and analyze measured physiological signals and to command administration of said optimal pulse stimulating procedure to said control panel [20];
d. said at least one MAC unit configured to analyze measured physiological signals as feedback signals for monitoring said applied pulse stimulating procedure, and command said control panel [20] accordingly;
e. said control panel [20] and said at least one MAC unit are configured to implement said stimulating procedures in intervals which range from about 0 and about 300 [sec];
f. said control panel [20] and said at least one MAC unit are configured to implement said pulse stimulating procedure to block the temperature signals traveling from the entire body or selected body parts, from reaching to the brain, such that the thermoregulation system reaction to changes in skin or core temperature is isolated;
g. said control panel [20] and said at least one MAC unit are configured to implement said stimulating procedure to control and modulate said body's thermoregulation system for at least one of the following:
i) maintain normo-thermia or sensation of normo-thermia,
ii) either induce or maintain hypo-thermia or sensation of hypo-thermia, and
iii) either induce or maintain hyper-thermia or sensation of hyper-thermia;
h. said control panel [20] and said at least one MAC unit are configured to implement said stimulating procedure to control and modulate said thermoregulation system physiological responses including: vasodilatation, vasoconstriction of blood vessels, shivering and/or heart rate, by affecting the efferent neurological pathways;
i. said at least one pair of stimulating electrodes [5] are configured for predetermined location selected from a group consisting of: topically close to the spinal cord, remote from said spinal cord, transcutaneously, or by means of minimally invasive means to said spinal cord, and any combination thereof;
j. said at least one pair of stimulating electrodes [5] are configured to be attached to said body using at least one patch [6];
k. said at least one pair of stimulating electrodes [5] are further configured to operate as sensors for measuring Electro-Cardio-Graph (ECG) signals;
l. said least one pair of stimulating electrodes [5] are further configured to operate as sensors for measuring Neuro-Electric signals;
m. said control panel [20] and said MAC units are configured to command said stimulation procedure along the whole length of the spinal cord or selected segments thereof, according to required treatment and clinical need;
n. said control panel [20] and said MAC units are configured to command and implement said stimulation procedure for controlling said body's temperature and thermoregulation system for the entire body or selected body parts; said at least one MAC unit is configured to command said stimulation procedure to simultaneously control said selected body parts, each for normo-thermia, hyper-thermia or hypo-thermia;
o. said system [1] further comprises means for application of medication selected from a group consisting of: local or general anesthetics, analgesics and or sedation agents, muscle blocking or relaxants agents, pain blocking agents, consciousness impairing agents, and any combination thereof;
p. said control panel [20] configured to control said application of stimulation procedure automatically;
q. said control panel [20] configured to control said application of stimulation procedure at least partially manually;
r. said display monitor [30] is configured to display parameters selected from a group consisting of: said at least one MAC unit measurements, said at least one MAC unit analysis results, said at least one MAC unit command for said stimulating procedures, said control panel [20] optional said stimulating procedures, said control panel [20] administrated said stimulating procedure, and any combination thereof;
s. said control panel [20] and said display monitor [30] are combined into a single device, such as a touch screen;
t. said control panel [20], said display monitor [30] and said pulse generator [10] are combined into a single device;
u. said at least two said MAC units are activated simultaneously, said control panel [20] consequent upon the first MAC unit that requires a change in generation of said pulse stimulating procedure;
v. said control panel [20] and said at least one MAC unit are configured to command at least one predetermined pulse stimulation procedure selected from the group consisting of:
i) stimulation of the fast conduction Aβ fibers, such that the “thermal input” transferred to the hypothalamus is modulated,
ii) stimulation of said Aβ fibers to control the passage of sensory information travels to said hypothalamus,
iii) stimulation of the slow conduction C fibers for a “heat” input, such that said thermal input received by said hypothalamus is modulated,
iv) stimulation of said C fibers to block said “heat” input, such that said thermal input received by said hypothalamus is modulated and the generation of a new action potential is avoided,
v) stimulation of the medium conduction Aδ fibers for a “cold” input message, such that said thermal input received by said hypothalamus is modulated,
vi) stimulation of said Aδ fibers for blocking the “cold” input message, such that said thermal input received by said hypothalamus is modulated and new action potential to be generated is avoided,
vii) stimulation or blockage of efferent motor neurons receiving input from the thermoregulation system, related to changes in body temperature, such that said motor neurons activity is modulated,
viii) stimulation of the nerve fibers to simulate “cold” or “heat” input, from different parts of said body, and
ix) any combination thereof;

111. The system [1] according to claim 109, wherein said control panel [20] and said at least one MAC unit regulate said stimulating procedures of electric pulses by means of current's amplitude [A], frequency [F] and phase [φ], applied to said at least one channel of electrical and/or electromagnetic stimuli; further wherein at least one of the following holds true:
a. said control panel [20] and said at least one MAC unit are configured to regulate said frequency [F] in the range of about 0 and about 30,000 [Hz];
b. said control panel [20] and said at least one MAC unit are configured to regulate said frequency [F] in the range of about 0 and about 1000 [Hz];
c. said control panel [20] and said at least one MAC unit are configured to implement said stimulating procedure in cycles with different said frequency [F] for each cycle; and
d. said control panel [20] and said at least one MAC unit are configured to regulate said frequency [F] in a manner selected from the group consisting of: linear, nonlinear, sinusoidal, and any combination thereof.

112. The system [1] according to claim 109, wherein said at least one MAC unit is selected from a group consisting of:
a. temperature (T) unit [70], configured to measure skin and/or core temperature,
b. Heart Rate Variability (HRV) unit [50], configured to measure heart beat,
c. Electromyography (EMG) unit [60], configured to measure muscles electrical activity,
d. oxygen utilization (Vo2) measuring unit [80], configured to measure oxygen utilization,
e. neuro-signal unit [90], configured to measure neuro-electric activity, and
f. any combination thereof.

113. The system [1] according to claim 112, wherein at least one of the following holds true:
a. said HRV unit [50] further comprises: an HRV receiving unit [51], an HRV analysis unit [52], an HRV activating unit [53] and an HRV output pulse generator [59];
b. said EMG unit [60] further comprises: an EMG receiving unit [61], an EMG analysis unit [62], an EMG activating unit [63], an EMG identification unit [65] and an EMG output pulse generator [69];
c. said temperature measuring unit [70] further comprises: a skin receiving unit [71s], a core receiving unit [71c], a temperature measuring analysis unit [62], a temperature measuring activating unit [73] and a temperature measuring output pulse generator [79]; and
d. said oxygen utilization (Vo2) measuring unit [80] further comprises: a Vo2 receiving unit [81], a Vo2 analysis unit [82], a Vo2 activating unit [83] and a Vo2 output pulse generator [89]; The system [1] according to claim 3, wherein said neuro-signal unit [90] further comprises: a neuro-signal receiving unit [91], a neuro-signal analysis unit [92], a neuro-signal activating unit [93], a neuro-signal identification unit [95], a neuro-signal summing unit [96] and a neuro-signal output pulse generator [99].

114. The system [1] according to claim 109, wherein said system [1] further comprises at least one Heat Exchange Unit (HEU) [100] for heating or cooling said body.

115. The system [1] according to claim 114, wherein said HEU [100] comprises: at least one thermal sensor [101], an HEU analysis unit [102], an HEU activating unit [103] and at least one of the following: an HEU output to heaters unit [109] and at least one heater [105], an HEU output to coolers unit and at least one cooler.

116. The system [1] according to claim 115, wherein said heaters [105] or said coolers are placed on a body part selected from the group consisting of: hand palms, feet soles, wrapped around large body parts, and any combination thereof.

117. The system [1] according to claim 109, wherein said least one pair of stimulating electrodes [5] are configured to be placed parallel to the vertebral column and over the intervertebral foramen, such that stimulation of the appropriate roots of spinal nerves, which supply the affected dermatome, are enabled.

118. The system [1] according to claim 117, wherein said least one pair of stimulating electrodes [5] layout in relation to the spinal cord vertebrates in a configuration selected from a group consisting of:
a. in a perpendicular manner, between said vertebrates as shown in FIG. 10 A;
b. in a perpendicular manner, across said vertebrates as shown in FIG. 10 B;
c. in a cross diagonally manner, where one electrode is near one vertebrate and the other electrode is near the second following vertebrate, as shown in FIG. 10 C;
d. in a cross diagonally manner, between said vertebrates, where one is close to said spine and the other is far from said spine, as shown in FIG. 10 D;
e. in a cross diagonally manner, between the vertebrates, as shown in FIG. 10 E;
f. in a cross diagonally manner, where one electrode is near one vertebrate and the other electrode is near the following vertebrate, as in FIG. 10 F;
g. in a cross diagonally manner, between said vertebrates, as shown in FIG. 10 G;
h. in a cross diagonally manner, between said vertebrates, where both said electrodes are on the same side of said spine, as shown in FIG. 10 H;
i. in a cross diagonally manner, where one electrode is near one vertebrate and the other electrode is far from the second following vertebrate, as shown in FIG. 10 I; and
j. any combination thereof.

119. A method for controlling and modulating a mammalian's body temperature and thermoregulation system, said method comprising steps of:
a. providing a system [1] for controlling and modulating a mammalian's body temperature and thermoregulation system, comprising:
i. a display monitor [30];
ii. a pulse generator [10];
iii. at least one channel of electrical or electromagnetic stimuli having at least one pair of stimulating electrodes [5], connected to said pulse generator [10], and configured for cutaneous application on said body's spinal cord, transcutaneously or by minimally invasive means;
iv. a control panel [20], for controlling said pulse generator [10] and said at least one pair of stimulating electrodes [5]; and
v. at least one Measurement Analysis and Command (MAC) unit for measuring physiological parameters, analyzing their signals and commanding an optimal pulse stimulating procedure to said control panel [20];
b. placing said electrodes [5] over the skin bilaterally along said body's back;
c. measuring said physiological parameters;
d. receiving said measured physiological parameters;
e. analyzing said measured physiological parameters;
f. determining whether a change in the parasympathetic activity in the body requires activating said pulse generator [10], due to a change in the patient's body temperature;
g. determining which of said electrodes [5], placed on which dermatome, should be stimulated;
h. determining an optimal stimulation procedure;
i. stimulating said optimal stimulation procedure, at below pain threshold level, thereby inducing sense of heat or coldness;
j. stimulating said optimal stimulation procedure, for covering all somatic dermatomes, such that all incoming thermal data is subject to filtering or modulation, and
k. monitoring said measured physiological parameters as feedback signals for monitoring said applied stimulating procedure, and commanding said control panel [20] accordingly;
wherein said optimal pulse stimulating procedure is configured to implement said optimal stimulating procedure to afferent neural pathways involved in said thermoregulation system, thereby controlling and modulating said body's temperature and thermoregulation system.

120. The method according to claim 119, wherein at one of the following holds true:
a. further comprising a step of selecting said optimal stimulating procedure form a group consisting of:
i) blocking said afferent neural pathways,
ii) evoking said afferent neural pathways,
iii) attenuating said afferent neural pathways, and
iv) any combination thereof;
b. further comprising a step of selecting said a pulse generator [10] from a group consisting of:
i) Trans Electrical Neuro Stimulation (TENS),
ii) Interferential Current (IFC),
iii) Electro Magnetic stimulation (EMS), and
iv) any combination thereof;
c. said at least one MAC unit configured for receiving and analyzing measured physiological signals and for commanding administration of said optimal pulse stimulating procedure to said control panel [20];
d. said at least one MAC unit configured for analyzing measured physiological signals as feedback signals for monitoring said applied pulse stimulating procedure, and commanding said control panel [20] accordingly;
e. said control panel [20] and said at least one MAC unit are configured for implementing said stimulating procedures in intervals which range from about 0 and about 300 [sec];
f. said control panel [20] and said at least one MAC unit are configured for implementing said pulse stimulating procedure for blocking the temperature signals traveling from the entire body or selected body parts, from reaching to the brain, thereby isolating the thermoregulation system reaction to changes in skin or core temperature;
g. said control panel [20] and said at least one MAC unit are configured for implementing said stimulating procedure for controlling and modulating said body's thermoregulation system for at least one of the following:
i) maintaining normo-thermia or sensation of normo-thermia,
ii) either inducing or maintaining hypo-thermia or sensation of hypo-thermia, and
iii) either inducing or maintaining hyper-thermia or sensation of hyper-thermia;
h. said control panel [20] and said at least one MAC unit are configured for implementing said stimulating procedure for controlling and modulating said thermoregulation system physiological responses including: vasodilatation, vasoconstriction of blood vessels, shivering and/or heart rate, by affecting the efferent neurological pathways;
i. said at least one pair of stimulating electrodes [5] are configured for predetermined location selected from a group consisting of: topically close to the spinal cord, remote from said spinal cord, transcutaneously, or by means of minimally invasive means to said spinal cord, and any combination thereof;
j. said at least one pair of stimulating electrodes [5] are configured for being attached to said body using at least one patch [6];
k. said at least one pair of stimulating electrodes [5] are further configured for operating as sensors for measuring Electro-Cardio-Graph (ECG) signals;
l. said least one pair of stimulating electrodes [5] are further configured for operating as sensors for measuring Neuro-Electric signals;
m. said control panel [20] and said MAC units are configured for commanding said stimulation procedure along the whole length of the spinal cord or selected segments thereof, according to required treatment and clinical need;
n. said control panel [20] and said MAC units are configured for commanding and implement said stimulation procedure for controlling said body's temperature and thermoregulation system for the entire body or selected body parts; said at least one MAC unit is configured for commanding said stimulation procedure for simultaneously controlling said selected body parts, each for normo-thermia, hyper-thermia or hypo-thermia;
o. further providing said system [1] with means for application of medication and selected from a group consisting of: local or general anesthetics, analgesics and or sedation agents, muscle blocking or relaxants agents, pain blocking agents, consciousness impairing agents, and any combination thereof;
p. said control panel [20] configured for controlling said application of stimulation procedure automatically;
q. said control panel [20] configured for controlling said application of stimulation procedure at least partially manually;
r. said display monitor [30] is configured for displaying parameters selected from a group consisting of: said at least one MAC unit measurements, said at least one MAC unit analysis results, said at least one MAC unit command for said stimulating procedures, said control panel [20] optional said stimulating procedures, said control panel [20] administrated said stimulating procedure, and any combination thereof;
s. further comprising a step of combining said control panel [20] and said display monitor [30] to a single device, such as a touch screen;
t. further comprising a step of combining said control panel [20], said display monitor [30] and said pulse generator [10] to a single device;
u. further comprising a step of simultaneously activating said at least two said MAC units, said control panel [20] consequent upon the first MAC unit that requires a change in generation of said pulse stimulating procedure;
v. said control panel [20] and said at least one MAC unit are configured for commanding at least one predetermined pulse stimulation procedure selected from the group consisting of:
i) stimulating the fast conduction Aβ fibers, thereby modulating the “thermal input” transferred to the hypothalamus,
ii) stimulating the said Aβ fibers to control the passage of sensory information travels to said hypothalamus,
iii) stimulating the slow conduction C fibers for a “heat” input, thereby modulating said thermal input received by said hypothalamus,
iv) stimulating said C fibers to block said “heat” input, thereby modulating said thermal input received by said hypothalamus is modulated and avoiding the generation of a new action potential,
v) stimulating the medium conduction Aδ fibers for a “cold” input message, thereby modulating said thermal input received by said hypothalamus,
vi) stimulating said Aδ fibers for blocking the “cold” input message, thereby modulating said thermal input received by said hypothalamus and avoiding the generation of new action potential,
vii) stimulating or blocking of efferent motor neurons receiving input from the thermoregulation system, related to changes in body temperature, thereby modulating said motor neurons activity,
viii) stimulating the nerve fibers to simulate “cold” or “heat” input, from different parts of said body, and
ix) any combination thereof;

121. The method according to claim 119, further comprising step of regulating said stimulating procedures of electric pulses, by means of current's amplitude [A], frequency [F] and phase [φ], applied to said at least one channel of electrical and/or electromagnetic stimuli; further wherein at least one of the following holds true:
a. said control panel [20] and said at least one MAC unit are configured for regulating said frequency [F] in the range of about 0 and about 30,000 [Hz];
b. said control panel [20] and said at least one MAC unit are configured for regulating said frequency [F] in the range of about 0 and about 1000 [Hz];
c. said control panel [20] and said at least one MAC unit are configured for implementing said stimulating procedure in cycles with different said frequency [F] for each cycle; and
d. said control panel [20] and said at least one MAC unit are configured for regulating said frequency [F] in a manner selected from the group consisting of: linear, nonlinear, sinusoidal, and any combination thereof.

122. The method according to claim 119, further comprising step of selecting said at least one MAC unit from a group consisting of:
a. temperature (T) unit [70], configured to measure skin and/or core temperature,
b. Heart Rate Variability (HRV) unit [50], configured to measure heart beat,
c. Electromyography (EMG) unit [60], configured to measure muscles electrical activity,
d. oxygen utilization (Vo2) measuring unit [80], configured to measure oxygen utilization,
e. neuro-signal unit [90], configured to measure neuro-electric activity, and
f. any combination thereof.

123. The method according to claim 122, wherein at least one of the following holds true:
a. said HRV unit [50] further comprises: an HRV receiving unit [51], an HRV analysis unit [52], an HRV activating unit [53] and an HRV output pulse generator [59];
b. said EMG unit [60] further comprises: an EMG receiving unit [61], an EMG analysis unit [62], an EMG activating unit [63], an EMG identification unit [65] and an EMG output pulse generator [69];
c. said temperature measuring unit [70] further comprises: a skin receiving unit [71s], a core receiving unit [71c], a temperature measuring analysis unit [62], a temperature measuring activating unit [73] and a temperature measuring output pulse generator [79]; and
d. said oxygen utilization (Vo2) measuring unit [80] further comprises: a Vo2 receiving unit [81], a Vo2 analysis unit [82], a Vo2 activating unit [83] and a Vo2 output pulse generator [89]; The system [1] according to claim 3, wherein said neuro-signal unit [90] further comprises: a neuro-signal receiving unit [91], a neuro-signal analysis unit [92], a neuro-signal activating unit [93], a neuro-signal identification unit [95], a neuro-signal summing unit [96] and a neuro-signal output pulse generator [99].

124. The method according to claim 119, further comprising step of providing said system [1] with at least one Heat Exchange Unit (HEU) [100] for heating or cooling said body; further wherein said HEU [100] comprises: at least one thermal sensor [101], an HEU analysis unit [102], an HEU activating unit [103] and at least one of the following: an HEU output to heaters unit [109] and at least one heater [105], an HEU output to coolers unit and at least one cooler.

125. The method according to claim 124, further comprising step of placing said heaters [105] and/or said coolers on a body part selected from the group consisting of: hand palms, feet soles, wrapped around large body parts, and any combination thereof.

126. The method according to claim 119, wherein said least one pair of stimulating electrodes [5] are configured for being placed parallel to the vertebral column and over the intervertebral foramen, thereby enabling stimulation of the appropriate roots of spinal nerves which supply the affected dermatome.

127. The method according to claim 126, further comprising step of laying out said least one pair of stimulating electrodes [5] in relation to the spinal cord vertebrates and in a configuration selected from a group consisting of:
a. in a perpendicular manner, between said vertebrates as shown in FIG. 10 A;
b. in a perpendicular manner, across said vertebrates as shown in FIG. 10 B;
c. in a cross diagonally manner, where one electrode is near one vertebrate and the other electrode is near the second following vertebrate, as shown in FIG. 10 C;
d. in a cross diagonally manner, between said vertebrates, where one is close to said spine and the other is far from said spine, as shown in FIG. 10 D;
e. in a cross diagonally manner, between the vertebrates, as shown in FIG. 10 E;
f. in a cross diagonally manner, where one electrode is near one vertebrate and the other electrode is near the following vertebrate, as in FIG. 10 F;
g. in a cross diagonally manner, between said vertebrates, as shown in FIG. 10 G;
h. in a cross diagonally manner, between said vertebrates, where both said electrodes are on the same side of said spine, as shown in FIG. 10 H;
i. in a cross diagonally manner, where one electrode is near one vertebrate and the other electrode is far from the second following vertebrate, as shown in FIG. 10 I; and
j. any combination thereof.

128. A system configured to control and modulate a mammalian's body temperature and thermoregulation system, comprising:
a. a display monitor [30];
b. a pulse generator [10];
c. at least one channel of electrical and/or electromagnetic stimuli having at least one pair of stimulating electrodes [5], connected to said pulse generator [10], and configured for cutaneous application on said body's spinal cord, transcutaneously and/or by minimally invasive means;
d. at least one measurement unit configured to measure physiological parameters, and
e. a control panel [20], configured to receive and analyze said physiological signals, and further to implement and command an optimal pulse stimulating procedure to said pulse generator [10], thereby control said pulse generator [10] and said at least one pair of stimulating electrodes [5];
wherein said control panel [20] is configured to implement said optimal pulse stimulating procedure to the afferent neural pathways involved in said thermoregulation system; said optimal stimulating procedure is selected form a group consisting of:
i) blocking said afferent neural pathways,
ii) evoking said afferent neural pathways,
iii) attenuating said afferent neural pathways, and
iv) any combination thereof;
such that said body's temperature and thermoregulation system are controlled and modulated.
